(19) United States
(12) Patent Application Publication
(10) Pub. No.: US 2005/0004196 A1
(43) Pub. Date: Jan. 6, 2005

(54) Pharmaceutical compositions including an ether and selective COX-2 inhibitor and methods for using such

(75) Inventor: Mark C. Kowala, Ann Arbor, MI (US)

Correspondence Address: WARNER-LAMBERT COMPANY, 2800 PLYMOUTH RD, ANN ARBOR, MI, 48105, US

(73) Assignee: WARNER-LAMBERT COMPANY LLC

(21) Appl. No.: 10/884,891

(22) Filed: Jul. 2, 2004

Related U.S. Application Data
(60) Provisional application No. 60/484,808, filed on Jul. 3, 2003.

(51) Int. Cl. A61K 31/41; A61K 31/19; A61K 31/11

(52) U.S. Cl. 514/381; 514/574; 514/693

(57) ABSTRACT

Disclosed herein are pharmaceutical compositions including a dialkyl ether, substituted alkyl, substituted aryl-alkyl, substituted dialkyl thioether, substituted dialkyl ketone, substituted-alkyl, or a pharmaceutically acceptable salt of said dialkyl ether, substituted alkyl, substituted aryl-alkyl, substituted dialkyl thioether, substituted dialkyl ketone, or substituted-alkyl, and a selective cyclooxygenase-2 (COX-2) inhibitor, or a pharmaceutically acceptable salt of said selective COX-2 inhibitor. Also disclosed are methods of using such pharmaceutical compositions for the treatment of inflammation and inflammation-associated diseases, inflammation and inflammation-associated disorders mediated by proinflammatory cytokines, and proinflammatory cytokine induced CRP production.

### CROSS-REFERENCE TO RELATED APPLICATIONS

This application claims benefit from U.S. Provisional Patent Application No. 60/484,808, filed Jul. 3, 2003.

### BACKGROUND OF THE INVENTION

This invention relates to pharmaceutical compositions including a dialkyl ether, substituted alkyl, substituted aryl-alkyl, substituted dialkyl thioether, substituted dialkyl ketone, substituted-alkyl, or a pharmaceutically acceptable salt of said dialkyl ether, substituted alkyl, substituted aryl-alkyl, substituted dialkyl thioether, substituted dialkyl ketone, or substituted-alkyl, and a selective cyclooxygenase-2 (COX-2) inhibitor, or a pharmaceutically acceptable salt of said selective COX-2 inhibitor. This invention further relates to methods of using such pharmaceutical compositions for the treatment of inflammation and inflammation-associated diseases.

Nonsteroidal anti-inflammatory drugs (NSAID's) are widely used because of their analgesic and anti-inflammatory activity. It is accepted that common NSAID's work by blocking the activity of cyclooxygenase (COX), also known as prostaglandin G/H synthase (PGHS), the enzyme that converts arachidonic acid into prostanoids. Prostaglandins, especially prostaglandin E₂ (PGE₂), which is the predominant eicosanoid detected in inflammation conditions, are mediators of pain, fever and other symptoms associated with inflammation. Inhibition of the biosynthesis of prostaglandins has been a therapeutic target of anti-inflammatory drug discovery. The therapeutic use of conventional NSAID's is, however, limited due to drug associated side effects, including life threatening ulceration and renal toxicity. An alternative to NSAID's is the use of corticosteroids; however, long-term therapy can also result in severe side effects.

Two forms of cyclooxygenase (COX) known to exist: COX-1 and COX-2, the former being a constitutive form and the latter being an inducible form. COX-1 exists in the stomach, intestines, kidneys and platelets while COX-2 is expressed during inflammation. Both COX enzyme isoforms metabolize arachidonic by a similar mechanism, but each have different substrate specificities. Selective COX-2 inhibitors are advantageous in the treatment of pain and inflammation while avoiding such side effects as gastric and renal toxicity.

C-reactive protein (CRP) is a marker of systemic inflammation (i.e., levels of CRP correlate with the level of systemic inflammation in an individual). CRP is produced mainly in the liver in response to proinflammatory cytokines, as part of an acute phase response. Increased levels of CRP have been independently associated with coronary heart disease, metabolic syndrome, type II diabetes melitis, glucose intolerance, hypertension, osteoarthritis or systemic inflammatory diseases such as rheumatoid arthritis, psoriatic arthritis, spondyloarthropathy or vasculitis.

Certain carboxyalkylethers are effective in lowering plasma concentrations of CRP, as is described in U.S. Provisional Patent Application 60/426,565, U.S. patent application Ser. No. 10/712,859, and PCT International Patent Application Number PCT/IB03/04930, which are incorporated by reference herein in their entirety.

Because inflammation and inflammation-associated diseases are prevalent throughout the world, the need continues to develop new and improved treatments, as well as agents that will actually prevent the formation of these diseases.

We have now discovered that treatment and prevention of inflammation and inflammation-associated diseases can be effected by co-administering an effective amount effective amount of a substituted dialkyl ether, substituted aryl-alkyl ether, substituted dialkyl thioether, substituted dialkyl ketone, or substituted-alkyl compound, or a pharmaceutically acceptable salt thereof, and a COX-2 inhibitor.

### SUMMARY OF THE INVENTION

Generally the present invention relates to methods and pharmaceutical compositions including a dialkyl ether, substituted alkyl, substituted aryl-alkyl, substituted dialkyl thioether, substituted dialkyl ketone, substituted-alkyl, or a pharmaceutically acceptable salt of said dialkyl ether, substituted alky, substituted aryl-alkyl, substituted dialkyl thioether, substituted dialkyl ketone, or substituted-alkyl, and a selective COX-2 inhibitor, or a pharmaceutically acceptable salt of said selective COX-2 inhibitor.

One embodiment is a pharmaceutical composition including

a.) a substituted dialkyl ether compound of Formula I 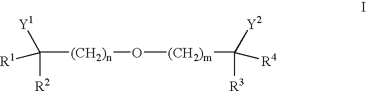
or a pharmaceutically acceptable salt thereof,
where:
n and m independently are integers of from 2 to 9;
R¹, R², R³, and R⁴ independently are C₁-C₆ alkyl, C₂-C₆ alkenyl, or C₂-C₆ alkynyl; or
   R¹ and R² together with the carbon atom to which they are attached, or R³ and R⁴ together with the carbon atom to which they are attached, or R¹ and R² together with the carbon atom to which they are attached and R³ and R⁴ together with the carbon atom to which they are attached, can complete a carbocyclic ring having from 3 to 6 carbons;
Y¹ and Y² independently are COOH, CHO, tetrazole, or COOR⁵, wherein R⁵ is C₁-C₆ alkyl, C₂-C₆ alkenyl, or C₂-C₆ alkynyl; and
where the alkyl, alkenyl, and alkynyl groups may be substituted with one or two groups selected from halo, hydroxy, C₁-C₆ alkoxy, and phenyl;
b.) a selective COX-2 inhibitor or a pharmaceutically acceptable salt thereof; and
c.) a pharmaceutically acceptable carrier or diluent.
   Another embodiment is a method of treating or preventing inflammation or an inflammation-associated disorder in a subject including co-administering to the subject having or susceptible to such inflammation or inflammation-associated disorder, a therapeutically-effective amount of a COX-2 inhibitor or a pharmaceutically acceptable salt thereof and a therapeutically-effective amount of a substituted dialkyl ether compound of Formula I 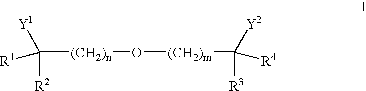
or a pharmaceutically acceptable salt thereof,
wherein:
n and m independently are integers of from 2 to 9;
R¹, R², R³, and R⁴ independently are C₁-C₆ alkyl, C₂-C₆ alkenyl, or C₂-C₆ alkynyl; or
   R¹ and R² together with the carbon atom to which they are attached, or R³ and R⁴ together with the carbon atom to which they are attached, or R¹ and R² together with the carbon atom to which they are attached and R³ and R⁴ together with the carbon atom to which they are attached, can complete a carbocyclic ring having from 3 to 6 carbons;
Y¹ and Y² independently are COOH, CHO, tetrazole, or COOR⁵, wherein
R⁵ is C₁-C₆ alkyl, C₂-C₆ alkenyl, or C₂-C₆ alkynyl; and
   wherein the alkyl, alkenyl, and alkynyl groups may be substituted with one or two groups selected from halo, hydroxy, C₁-C₆ alkoxy, and phenyl.

### DESCRIPTION OF THE DRAWINGS

FIG. 1 (“FIG. 1”) is a bar graph showing the effect of 6-(5-carboxy-5-methyl-hexyloxy)-2,2-dimethyl-hexanoic acid, calcium salt (“CI-1027”) at a dose of 10 mg/kg, rofecoxib (Vioxx®) (3 mg/kg) and CI-1027/rofecoxib combination on the change in rat hind paw weight distribution, (expressed in grams) in rats with monosodium iodoacetate (“MIA”)-induced knee joint arthritis. Arthritis was induced on Day 0 by injection of a physiologic saline solution of 1 mg of MIA through the infrapatellar ligament of the right knee. On day 14, baseline readings were obtained followed by oral administration of either vehicle, CI-1027, VIOXX® (Merck & Co.) or CI-1027/VIOXX® combination. Changes in hind paw weight distribution data were measured at 2 hours post compound administration.

### DETAILED DESCRIPTION OF THE INVENTION

We have discovered that the co-administration of a substituted dialkyl ether, substituted aryl-alkyl ether, substituted dialkyl thioether, substituted dialkyl ketone, or substituted-alkyl compound, or a pharmaceutically acceptable salt thereof, and a COX-2 inhibitor, or a pharmaceutically acceptable salt thereof, is effective in the treatment and prevention of inflammation and inflammation-associated disorders. An appreciation of various aspects of the invention will be gained through the following discussion and the examples provided below.

For purposes of the subject invention, unless otherwise noted, the terms “inflammation” and “inflammation-associated” refer to any and all such inflammatory reactions including, but not limited to, immune-related responses and/or allergic reactions to a physical, chemical, or biological stimuli. Such inflammation-associated diseases include for example, osteoarthritis, rheumatoid arthritis, osteoarthritic joint pain, rheumatoid arthritic joint, joint pain, inflammatory pain, acute pain, chronic pain, and cartilage damage.

It should be appreciated that the terms “uses”, “utilizes”, and “employs”, and their derivatives thereof, are used interchangeably when describing an aspect of an invention method, composition, or combination.

The terms “including,” “having,” and “containing” are open ended unless otherwise indicated.

The phrase “admixed” or “in admixture” means the ingredients so mixed comprise either a heterogeneous or homogeneous mixture. In some circumstances a homogeneous mixture is preferred. In other circumstances, a heterogeneous mixture is preferred.

The phrase combination therapy (or adjunct therapy or co-administration) in defining the use of a selective COX-2 inhibitor, or a pharmaceutically acceptable salt thereof, and a substituted dialkyl ether, substituted aryl-alkyl ether, substituted dialkyl thioether, substituted dialkyl ketone, or substituted-alkyl compound, or a pharmaceutically acceptable salt thereof, is intended to embrace administration of each agent in a sequential manner in a regimen that will provide beneficial effects of the drug combination, and is intended as well to embrace the administration of these agents in a substantially simultaneous manner, such as in a single formulation having a fixed ratio of these active agents, or in multiple, separate formulations for each agent.

The term “ED₄₀” means the dose of a drug, including an active compound, or a pharmaceutically acceptable salt thereof, that is sufficient to treat or prevent inflammation and inflammation-associated diseases, in at least 40% of the patients being treated.

The term “drug”, which is synonymous with the phrases “therapeutic agent”, “active component”, “active compound”, and “active ingredient”, includes a nontoxic therapeutic agent such as a selective COX-2 inhibitor, or a pharmaceutically acceptable salt thereof, and a substituted dialkyl ether, substituted aryl-alkyl ether, substituted dialkyl thioether, substituted dialkyl ketone, or substituted-alkyl compound, or a pharmaceutically acceptable salt thereof.

The term “nontoxic” means the efficacious dose is 10 times or greater than the dose at which a toxic effect is observed in 10% or more of a patient population.

The term “patient” means a mammal, and the two terms are used interchangeably herein.

For the purposes of this invention, the term “mammal” includes humans, companion animals such as cats and dogs, livestock animals such as horses, cows, pigs, goats, and sheep, and laboratory animals such as guinea pigs, rabbits, rats, mice, hamsters, and monkeys, and transgenic variants thereof. A human patient is preferred. Also preferred are companion animals, particularly dogs, cats, and horses. Also preferred are laboratory animals, particularly rabbits, rats, mice, and monkeys, and transgenic variants thereof.

For the purposes of this invention, the term “arthritis” includes osteoarthritis, rheumatoid arthritis, degenerative joint disease, spondyloarthropathies, gouty arthritis, systemic lupus erythematosus, juvenile arthritis, and psoriatic arthritis. The co-administration of a selective COX-2 inhibitor, or a pharmaceutically acceptable salt thereof, and a substituted dialkyl ether, substituted aryl-alkyl ether, substituted dialkyl thioether, substituted dialkyl ketone, or substituted-alkyl compound, or a pharmaceutically acceptable salt thereof, may also be useful for treating degenerative joint disease, spondyloarthropathies, gouty arthritis, systemic lupus erythematosus, juvenile arthritis, and psoriatic arthritis.

The phrase “cartilage damage” means a disorder of articular cartilage and subchondral bone characterized by hypertrophy of tissues in and around an involved joint, which may or may not be accompanied by deterioration of articular cartilage surface. As used herein, the phrase cartilage damage relates to damage to joint cartilage.

It should be appreciated that cartilage is a multicellular tissue found at joint linings and in other parts of the body, including the nose, for example. Cartilage tissue provides frictionless surfaces for joint movement, and structure and support for soft tissue features of the body such as the nostrils of the nose. When cartilage tissue is damaged by disease or trauma, breakdown products are formed and the physiological function of the tissue is impaired. There principally are three types of cartilage in a body, including articular cartilage.

The phrase “inhibiting cartilage damage” means the therapeutic effect of the co-administration of a selective COX-2 inhibitor, or a pharmaceutically acceptable salt thereof, and a substituted dialkyl ether, substituted aryl-alkyl ether, substituted dialkyl thioether, substituted dialkyl ketone, or substituted-alkyl compound, or a pharmaceutically acceptable salt thereof, that eliminates, alleviates, inhibits or prevents the onset of, inhibits the progress of, prevents further progress of, or reverses progression of, in part or in whole, any one or more pathological hallmarks or symptoms of cartilage damage observed for any of the diseases and disorders which have cartilage damage as a component of the disease or disorder pathology. A patient at risk for developing cartilage damage may be prophylactically treated just as a patient having cartilage damage may be medically treated.

It should be appreciated that a pathological hallmark of a disease or disorder relates to a structural change in a body that is a direct or indirect result of the body being afflicted with the disease or disorder. Such structural changes may be identified by clinical observation, examination of biopsied tissue, pathological examination or by imaging techniques such as X-ray or magnetic resonance imaging, of the affected structure. Illustrative examples of a pathological hallmark include histopathological damage to cartilage, thickening or thinning of bone, hypertrophy of muscle, fibrosis, a tear in a ligament or tendon, and the like.

The term “osteoarthritis” includes diseases of the joint principally characterized by the pathological hallmark of joint cartilage damage, and optionally the symptom of joint pain. Osteoarthritis patients typically do not suffer from inflammation of the joint, although they may experience transient inflammatory flares from time to time.

The term “rheumatoid arthritis” includes rheumatic diseases of the joint principally characterized by the symptom of joint inflammation, and optionally joint pain. Rheumatoid arthritis patients may eventually also experience damage to joint cartilage.

The term “treating” means the co-administration of a selective COX-2 inhibitor, or a pharmaceutically acceptable salt thereof, and a substituted dialkyl ether, substituted aryl-alkyl ether, substituted dialkyl thioether, substituted dialkyl ketone, or substituted-alkyl compound, or a pharmaceutically acceptable salt thereof, that eliminates, alleviates, inhibits or prevents the onset of, inhibits the progress of, prevents further progress of, or reverses progression of, in part or in whole, any one or more of the pathological hallmarks or symptoms of any one of the diseases and disorders being treated, including, but not limited to, the pathological hallmark of cartilage damage and the symptoms of pain and inflammation. A patient at risk for developing a disease or disorder may be prophylactically treated just as a patient having the disease or disorder may be medically treated.

The term “preventing” means prophylactic co-administration of a selective COX-2 inhibitor, or a pharmaceutically acceptable salt thereof, and a substituted dialkyl ether, substituted aryl-alkyl ether, substituted dialkyl thioether, substituted dialkyl ketone, or substituted-alkyl compound, or a pharmaceutically acceptable salt thereof, to an asymptomatic patient at risk for the disease or disorder being prevented to inhibit the onset of an associated pathological hallmark or symptom, including, but not limited to, the pathological hallmark of cartilage damage and the symptoms of pain and inflammation. Further, once onset of a pathological hallmark or symptom has begun, preventing means to prevent further progression or reverse progression, in part or in whole, of the pathological hallmark or symptom.

The term “improving” means co-administration of a selective COX-2 inhibitor, or a pharmaceutically acceptable salt thereof, and a substituted dialkyl ether, substituted aryl-alkyl ether, substituted dialkyl thioether, substituted dialkyl ketone, or substituted-alkyl compound, or a pharmaceutically acceptable salt thereof, that eliminates or prevents the loss, inhibits further loss, or improves, in part or in whole, of any one or more of the clinical measures of a function in a patient suffering from any one of the diseases and disorders being improved, including, but not limited rheumatoid arthritis and osteoarthritis.

The phrase “joint function” relates to any one or more of the clinical assessments of joint function, including stiffness, range of movement, flexibility, and movement-related symptoms (e.g., altered gait, pain, warmth, or inflammation), in a patient suffering from any one of the diseases and disorders being improved, including, but not limited the diseases of rheumatoid arthritis and osteoarthritis. The Western Ontario and McMaster Universities Osteoarthritis Index (“WOMAC”) may be used by a clinician to assess joint function.

The phrase “pain alleviating” means co-administration of a selective COX-2 inhibitor, or a pharmaceutically acceptable salt thereof, and a substituted dialkyl ether, substituted aryl-alkyl ether, substituted dialkyl thioether, substituted dialkyl ketone, or substituted-alkyl compound, or a pharmaceutically acceptable salt thereof, that eliminates, or inhibits or prevents onset of, suppresses, reduces, prevents, or otherwise inhibits, pain in a patient, including, but not limited to, the suppression, reduction, prevention, inhibition or elimination of pain symptoms due to cartilage damage, acute pain, chronic pain, mechanical pain, static allodynia, dynamic allodynia, bone cancer pain, headache, osteoarthritic pain, inflammatory pain, and pain associated with autoimmune disorders or fibromyalgia.

The phrase “joint pain” means any pain in a joint.

The phrase “osteoarthritic pain” means joint pain in an osteoarthritic joint.

The phrase “rheumatoid arthritic pain” means joint pain in a rheumatoid arthritic joint.

The phrase “inflammatory pain” means pain due to edema or swelling of any inflamed tissue, including inflammatory joint pain. Inflammatory joint pain includes rheumatoid arthritic pain.

The phrase “acute pain” means any pain, including, but not limited to, joint pain, osteoarthritic pain, rheumatoid arthritic pain, inflammatory pain, pain from a burn, pain from a cut, surgical pain, pain from fibromyalgia, bone cancer pain, menstrual pain, back pain, headache, static allodynia, and dynamic allodynia, that lasts from 1 minute to 91 days, 1 minute to 31 days, 1 minute to 7 days, 1 minute to 5 days, 1 minute to 3 days, 1 minute to 2 days, 1 hour to 91 days, 1 hour to 31 days, 1 hour to 7 days, 1 hour to 5 days, 1 hour to 3 days, 1 hour to 2 days, 1 hour to 24 hours, 1 hour to 12 hours, or 1 hour to 6 hours, per occurrence if left untreated. Acute pain includes, but is not limited to, joint pain, osteoarthritic pain, rheumatoid arthritic pain, inflammatory pain, pain from a burn, pain from a cut, surgical pain, pain from fibromyalgia, bone cancer pain, menstrual pain, back pain, headache, static allodynia, dynamic allodynia, acute joint pain, acute osteoarthritic pain, acute rheumatoid arthritic pain, acute inflammatory pain, acute headache, acute menstrual pain, acute back pain, and acute pain from fibromyalgia. Acute pain may be selected from acute joint pain, acute osteoarthritic pain, acute rheumatoid arthritic pain, acute inflammatory pain, acute headache, acute menstrual pain, and acute back pain. Acute pain may be selected from acute joint pain, acute osteoarthritic pain, acute rheumatoid arthritic pain, and acute inflammatory pain. Acute pain may be selected from acute joint pain, acute osteoarthritic pain, and acute rheumatoid arthritic pain. Acute pain may be selected from acute joint pain and acute osteoarthritic pain.

It should be appreciated that alleviating acute pain means having an appreciable pain alleviating effect within 91, 31, 7, 5, 3, or 2 days, or 24, 12, 6, 3, 2, 1, 0.5, 0.25, 0.20. 0.17, or 0.10 hours after co-administration of a selective COX-2 inhibitor, or a pharmaceutically acceptable salt thereof, and a substituted dialkyl ether, substituted aryl-alkyl ether, substituted dialkyl thioether, substituted dialkyl ketone, or substituted-alkyl compound, or a pharmaceutically acceptable salt thereof.

The phrase “chronic pain” means any pain, including, but not limited to, joint pain, osteoarthritic pain, rheumatoid arthritic pain, inflammatory pain, pain from a burn, pain from a cut, surgical pain, pain from fibromyalgia, bone cancer pain, menstrual pain, back pain, headache, static allodynia, dynamic allodynia, chronic joint pain, chronic osteoarthritic pain, chronic rheumatoid arthritic pain, chronic inflammatory pain, chronic headache, chronic back pain, and chronic pain from fibromyalgia that lasts longer than 91 days, 6 months, 1 year, 5 years, or 10 years per occurrence if left untreated. Chronic pain may be selected from chronic joint pain, chronic osteoarthritic pain, chronic rheumatoid arthritic pain, chronic inflammatory pain, chronic headache, chronic back pain, and chronic pain from fibromyalgia. Chronic pain may be selected from chronic joint pain, chronic osteoarthritic pain, chronic rheumatoid arthritic pain, chronic inflammatory pain, chronic headache, and chronic back pain. Chronic pain may be selected from chronic joint pain, chronic osteoarthritic pain, chronic rheumatoid arthritic pain, and chronic inflammatory pain. Chronic pain may be selected from chronic joint pain, chronic osteoarthritic pain, and chronic rheumatoid arthritic pain. Chronic pain may be selected from chronic joint pain and chronic osteoarthritic pain.

It should be appreciated that alleviating chronic pain means having an appreciable pain alleviating effect within 91, 60, 31, 28, 21, 14, 7, 3, or 2 days or 24, 12, 6, 3, 2, 1, 0.5, 0.25, 0.20. 0.17, or 0.10 hours after co-administration of a selective COX-2 inhibitor, or a pharmaceutically acceptable salt thereof, and a substituted dialkyl ether, substituted aryl-alkyl ether, substituted dialkyl thioether, substituted dialkyl ketone, or substituted-alkyl compound, or a pharmaceutically acceptable salt thereof.

It should be appreciated that pain substantially (>10%) resulting from a deficit of oxygen in an organ (e.g., brain, heart, or liver) is not embraced by any pain disclosed herein.

As used herein “proinflammatory cytokine induced CRP production” means CRP levels outside the liver or hepatocytes are increased in response to one or more proinflammatory cytokines. “Production” includes all increases in CRP levels regardless of the mechanism by which the level is increased. Mechanisms by which CRP levels are increased include, but are not limited to, secretion of CRP from the liver, increased transcription and/or translation of CRP and stability of CRP protein and/or mRNA.

The phrase “inflammation and inflammation-associated disorders mediated by proinflammatory cytokines” refers to inflammation and inflammation-associated disorders in a patient that are alleviated by administering to the patient an inhibitor of the cytokine IL-6, its cell-bound receptor IL-6 receptor, or IL-6sR, which is an unbound cleavage fragment of IL-6 receptor that can bind to IL-6 or IL-1β. An IL-6, IL-6sR, or IL-6 receptor inhibitor includes inhibitors of IL-6, IL-6sR, or IL-6 expression or biological activity and promoters of IL-6, IL-6sR, or IL-6 clearance, respectively.

The phrases “therapeutically effective amount” and “effective amount” are synonymous and mean an amount of a selective COX-2 inhibitor, or a pharmaceutically acceptable salt thereof, and a substituted dialkyl ether, substituted aryl-alkyl ether, substituted dialkyl thioether, substituted dialkyl ketone, or substituted-alkyl compound, or a pharmaceutically acceptable salt thereof, co-administered which is sufficient to alleviate, eliminate, inhibit or prevent the onset, or inhibit the progress, prevent further progress, or reverse progression, in part or in whole, of any one or more pathological hallmarks or symptoms of the disease or disorder that is appreciated or suspected or expected in the particular patient being treated.

It should be appreciated that a therapeutically effective or effective amount means an amount sufficient to have a desired effect in a patient to whom that amount has been administered. An illustrative example is where cartilage damage is being inhibited, a therapeutically effective amount includes a cartilage damage inhibiting effective amount. Where osteoarthritis is being treated, a therapeutically effective amount includes an osteoarthritis treating effective amount. Where pain is being alleviated, a therapeutically effective amount includes a pain alleviating effective amount. Where osteoarthritic or rheumatoid arthritic pain is being alleviated, a therapeutically effective amount includes an osteoarthritic or rheumatoid arthritic pain alleviating effective amount, respectively. In the context of a method for inhibiting proinflammatory cytokine induced CRP production, a therapeutically effective amount includes a proinflammatory cytokine induced CRP inhibitory amount.

Substituted dialkyl ether, substituted aryl-alkyl ether, substituted dialkyl thioether, substituted dialkyl ketone, or substituted-alkyl compounds useful in an invention method, composition, or combination include any aspect or embodiment of the therapeutic compounds described in U.S. Pat. Nos. 3,773,946; 3,930,024; 4,287,200; 4,689,344; 4,711,896; 5,648,387; 5,750,569; 5,756,544; 5,783,600; 6,410,802; 6,459,003; and 6,506,799; U.S. patent application Ser. Nos. 09/976,867; 09/976,938; 09/976,898; 09/976,899; and 10/205,939; U.S. Patent Application Publication Nos. US 2002/0077316; US 2003/0018013; US 2003/0022865; US 2003/0065195; and US 2003/0078239; and PCT International Application Publication numbers WO 96/30328; WO 98/30530; WO 00/59855; WO 01/55078; WO 02/30860; WO 02/30863; WO 02/30882; and WO 02/30884, which are each hereby incorporated herein by reference.

Examples of substituted dialkyl ethers useful in the present invention include those of Formula I 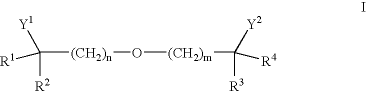
or a pharmaceutically acceptable salt thereof,
where:
n and m independently are integers of from 2 to 9;
R¹, R², R³, and R⁴ independently are C₁-C₆ alkyl, C₂-C₆ alkenyl, or C₂-C₆ alkynyl; or
   R¹ and R² together with the carbon atom to which they are attached, or R³ and R⁴ together with the carbon atom to which they are attached, or R¹ and R² together with the carbon atom to which they are attached and R³ and R⁴ together with the carbon atom to which they are attached, can complete a carbocyclic ring having from 3 to 6 carbons;
Y¹ and Y² independently are COOH, CHO, tetrazole, or COOR⁵, wherein R⁵ is C₁-C₆ alkyl, C₂-C₆ alkenyl, or C₂-C₆ alkynyl; and
where the alkyl, alkenyl, and alkynyl groups may be substituted with one or two groups selected from halo, hydroxy, C₁-C₆ alkoxy, and phenyl;
where halo includes chloro, bromo, and iodo, C₁-C₆ alkoxy is a C₁-C₆ alkyl group linked through oxygen.

Additional examples of substituted dialkyl ethers useful in the present invention include those of Formula I where n and m independently are integers of from 2 to 9; R¹, R², R³, and R⁴ independently are C₁-C₆ alkyl; and Y¹ and Y² independently are COOH or COOR⁵, wherein R⁵ is C₁-C₆ alkyl.

Other examples of substituted dialkyl ethers useful in the present invention include 6-(5-carboxy-5-methyl-hexyloxy)-2,2-dimethyl-hexanoic acid, represented by the structure drawn below:

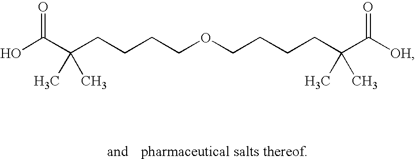

An example of a useful salt of 6-(5-carboxy-5-methyl-hexyloxy)-2,2-dimethyl-hexanoic acid is 6-(5-carboxy-5-methyl-hexyloxy)-2,2-dimethyl-hexanoic acid, calcium salt, represented by the structure drawn below:

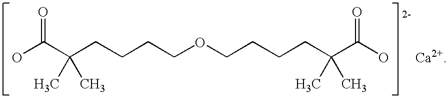

The substituted dialkyl ether named 6-(5-carboxy-5-methyl-hexyloxy)-2,2-dimethyl-hexanoic acid, calcium salt is known by other names, including “6-(5-carboxy-5-methyl-hexyloxy)-2,2-dimethyl-hexanoic acid, monocalcium salt,” “6-(5-carboxy-5-methyl-hexyloxy)-2,2-dimethyl-hexanoic acid, mono-calcium salt, “6,6′-oxybis(2,2-dimethylhexanoic acid),” “CI-1027” and gemcabene calcium.

It should be appreciated that the substituted dialkyl ether named 6-(5-carboxy-5-methyl-hexyloxy)-2,2-dimethyl-hexanoic acid, calcium salt may exist in a number of different physical forms, including Crystal Form 1 and Crystal Form 2. Crystal Form 1 and Crystal Form 2 of the substituted dialkyl ether named 6-(5-carboxy-5-methyl-hexyloxy)-2,2-dimethyl-hexanoic acid, calcium salt have been disclosed in PCT International Patent Application Publication No. WO 01/55078. The use of each of these crystal forms is within the scope of this invention method.

Crystal Form 1 has an x-ray powder diffraction pattern substantially comprising:

| # | 2-Theta | d(A) | Peak | P % | Area | Area % | FWHM |
|---|---|---|---|---|---|---|---|
| 1 | 6.760 | 13.0648 | 5106 | 100.0 | 1497 | 100.0 | 0.234 |
| 2 | 8.183 | 10.7953 | 1743 | 34.1 | 435 | 29.1 | 0.200 |
| 3 | 8.560 | 10.3207 | 1866 | 36.5 | 543 | 36.3 | 0.233 |
| 4 | 9.239 | 9.5638 | 234 | 4.6 | 29 | 1.9 | 0.096 |
| 5 | 9.760 | 9.0546 | 972 | 19.0 | 220 | 14.7 | 0.181 |
| 6 | 10.569 | 8.3634 | 156 | 3.1 | 12 | 0.8 | 0.061 |
| 7 | 11.141 | 7.9353 | 178 | 3.5 | 29 | 1.9 | 0.130 |
| 8 | 13.760 | 6.4304 | 266 | 5.2 | 46 | 3.1 | 0.138 |
| 9 | 15.599 | 5.6761 | 338 | 6.6 | 63 | 4.2 | 0.148 |
| 10 | 16.740 | 5.2917 | 433 | 8.5 | 64 | 4.3 | 0.118 |
| 11 | 17.420 | 5.0866 | 1890 | 37.0 | 689 | 46.0 | 0.291 |
| 12 | 20.639 | 4.3000 | 523 | 10.2 | 128 | 8.5 | 0.196 |
| 13 | 21.391 | 4.1505 | 188 | 3.7 | 20 | 1.3 | 0.085 |
| 14 | 22.139 | 4.0119 | 445 | 8.7 | 74 | 4.9 | 0.132 |
| 15 | 31.559 | 2.8326 | 270 | 5.3 | 24 | 1.6 | 0.070 |

Crystal Form 2 has an x-ray powder diffraction pattern substantially comprising:

| # | 2-Theta | d(A) | Peak | P % | Area | Area % | FWHM |
|---|---|---|---|---|---|---|---|
| 1 | 7.259 | 12.1686 | 9283 | 100.0 | 2482 | 100.0 | 0.214 |
| 2 | 8.739 | 10.1100 | 4191 | 45.1 | 603 | 24.3 | 0.115 |
| 3 | 9.386 | 8.9628 | 967 | 10.4 | 161 | 6.5 | 0.133 |
| 4 | 11.659 | 7.5838 | 430 | 4.6 | 49 | 1.9 | 0.089 |
| 5 | 13.955 | 6.3408 | 305 | 3.3 | 58 | 2.3 | 0.151 |
| 6 | 14.220 | 6.2233 | 326 | 3.5 | 73 | 2.9 | 0.178 |
| 7 | 15.387 | 5.7537 | 278 | 3.0 | 19 | 0.7 | 0.053 |
| 8 | 16.461 | 5.3806 | 986 | 10.6 | 187 | 7.5 | 0.152 |
| 9 | 17.361 | 5.1039 | 1490 | 16.1 | 348 | 14.0 | 0.187 |
| 10 | 18.063 | 4.9069 | 1284 | 13.8 | 323 | 13.0 | 0.201 |
| 11 | 19.302 | 4.5947 | 871 | 9.4 | 166 | 6.7 | 0.152 |
| 12 | 19.862 | 4.4664 | 686 | 7.4 | 142 | 5.7 | 0.166 |
| 13 | 20.200 | 4.3923 | 457 | 4.9 | 103 | 4.1 | 0.179 |
| 14 | 21.178 | 4.1918 | 656 | 7.1 | 97 | 3.9 | 0.117 |
| 15 | 21.641 | 4.1031 | 167 | 1.8 | 6 | 0.2 | 0.029 |
| 16 | 22.300 | 3.9833 | 794 | 8.6 | 192 | 7.7 | 0.193 |
| 17 | 23.218 | 3.8278 | 247 | 2.7 | 23 | 0.9 | 0.071 |
| 18 | 24.100 | 3.6897 | 183 | 2.0 | 34 | 1.3 | 0.145 |
| 19 | 25.481 | 3.4928 | 487 | 5.2 | 141 | 5.7 | 0.231 |
| 20 | 28.800 | 3.0974 | 134 | 1.4 | 14 | 0.6 | 0.083 |
| 21 | 29.297 | 3.0459 | 259 | 2.8 | 28 | 1.1 | 0.084 |
| 22 | 30.700 | 2.9099 | 287 | 3.1 | 20 | 0.8 | 0.055 |

It is well established in the art that unique crystal and polymorphic forms of compounds can be characterized by one or more unique 2θ values in the x-ray diffractogram. While several 2θ values have been recited above for Crystal Forms 1 and 2, a single 2θ value will suffice to identify a unique structure. For example Crystal Form 1 can be characterized, by the 2θ values 6.760 and 17.420 individually or together. Crystal Form 2 can be characterized, by the 2θ values 7.259 and 8.739 individually or together.

It should be appreciated that the substituted dialkyl ether named 6-(5-carboxy-5-methyl-hexyloxy)-2,2-dimethyl-hexanoic acid, calcium salt, may further exist as a hydrate, known by the name 6-(5-carboxy-5-methyl-hexyloxy)-2,2-dimethyl-hexanoic acid, monocalcium salt hydrate in PCT International Patent Application Publication No. WO 01/55078. The use of this or another hydrate form is within the scope of this invention method.

It should be appreciated that the substituted dialkyl ether named 6-(5-carboxy-5-methyl-hexyloxy)-2,2-dimethyl-hexanoic acid, calcium salt, may further exist as a C₁-C₁₂ alcohol solvate, including an ethyl alcohol, methanol, 1-propyl alcohol, 2-propyl alcohol, or 1-butyl alcohol solvate, known by the names 6-(5-carboxy-5-methyl-hexyloxy)-2,2-dimethyl-hexanoic acid, mono-calcium salt ethyl alcohol solvate, 6-(5-carboxy-5-methyl-hexyloxy)-2,2-dimethyl-hexanoic acid, mono-calcium salt methanol solvate, 6-(5-carboxy-5-methyl-hexyloxy)-2,2-dimethyl-hexanoic acid, monocalcium salt 1-propyl alcohol solvate, 6-(5-carboxy-5-methyl-hexyloxy)-2,2-dimethyl-hexanoic acid, monocalcium salt 2-propyl alcohol solvate, 6-(5-carboxy-5-methyl-hexyloxy)-2,2-dimethyl-hexanoic acid, monocalcium salt 1-butyl alcohol solvate, respectively, in PCT International Patent Application Publication No. WO 01/55078. The use of these and other alcohol solvate forms is within the scope of this invention method.

Further examples of dialkyl ethers of Formula I include
7,7′-oxybis(2,2-dimethylheptanoic acid);
5,5′-oxybis(2,2-dimethylpentanoic acid);
4,4′-oxybis(2,2-dimethylbutanoic acid);
8,8′-oxybis(2,2-dimethyloctanoic acid);
Ethyl 2,2-dimethyl-5-(4-methyl-4-ethoxycarbonylpentyloxy)pentanoate;
Ethyl 2,2-dimethyl-6-(5-methyl-5-ethoxycarbonylhexyloxy)hexanoate;
Methyl 2,2-dimethyl-8-(7-methyl-7-methoxycarbonyloctyloxy)octanoate;
7-(4-methyl-4-hydroxycarbonylpentyloxy)-2,2-dimethylheptanoic acid;
and a pharmaceutically acceptable salts thereof.

Yet further examples of dialkyl ethers of Formula I include
5-(3-Carboxy-3-methyl-butoxy)-2,2-dimethyl-pentanoic acid;
2,2-Diethyl-5-(4-methoxycarbonyl4-methyl-pentyloxy)-pentanoic acid;
6-(3-Carboxy-3-ethyl-4-methyl-pentyloxy)-2,2-diethyl-hexanoic acid methyl ester;
2-(3-Chloro-propyl)-5-(5-formyl-7-hydroxy-5-methyl-heptyloxy)-2-methyl-pentanoic acid;
6-(5-Carboxy-5-methyl-hexyloxy)-2,2-dimethyl-hexanoic acid;
6-(5-Carboxy-5-ethyl-heptyloxy)-2,2-diethyl-hexanoic acid, bis sodium salt;
6-(5-Butyl-5-methoxycarbonyl-nonyloxy)-2-ethyl-2-methyl-hexanoic acid;
6-(5-Ethoxycarbonyl-6-hydroxy-5-hydroxymethyl-hexyloxy)-2,2-bis-hydroxymethyl-hexanoic acid ethyl ester;
2,2-Dipropyl-6-[5-propyl-5-(1H-tetrazol-5-yl)-octyloxy]-hexanal;
1-{4-[4-(1-Carboxycyclopropan-1-yl)-butyloxy]-butyl}-cyclopropanecarboxylic acid;
1-[4-(5,5-Dimethyl-6-oxo-hexyloxy)-butyl]-cyclopentanecarbaldehyde;
2-Benzyl-6-(5,5-dimethyl-6-oxo-hexyloxy)-2-methyl-hexanal;
6-(6-Ethyl-6-formyl-octyloxy)-2,2-dimethyl-hexanoic acid;
7-(5-Carboxy-5-ethyl-6-methyl-heptyloxy)-2-ethyl-2-isobutyl-heptanoic acid;
2-[2-(6-Carboxy-6-hexyl-dodecyloxy)-ethyl]-2-hexyl-octanoic acid;
8-(3-Carboxy-3-isobutyl-5-methyl-hexyloxy)-2,2-dipropyl-octanoic acid, bis potassium salt;
8-(4-Carboxy-4-methyl-pentyloxy)-2,2-diethyl-octanoic acid;
2-Bromomethyl-9-(4-carboxy-4-chloromethyl-5-hydroxy-pentyloxy)-2-iodomethyl-nonanoic acid;
9-(5-Carboxy-5-pentyl-decyloxy)-2,2-bis-methoxymethyl-nonanoic acid, 1:1 salt with triethylamine;
10-(5,5-Dimethyl-6-oxo-hexyloxy)-2,2-dimethyl-decanoic acid;
11-(5-Hexyloxycarbonyl-5-methyl-hexyloxy)-2,2-dimethyl-undecanoic acid ethyl ester;
5-{3-Ethyl-11-[6-Ethyl-6-(1H-tetrazol-5-yl)-octan-1-yloxy]-undecan-3-yl}-tetrazole; and
11-(10-Benzyl-10-carboxy-11-chloro-undecyloxy)-2,2-diethyl-undecanoic acid;
and a pharmaceutically acceptable salts thereof.

Substituted dialkyl ethers of Formula I, and pharmaceutically acceptable salts thereof, including the compound named 6-(5-carboxy-5-methyl-hexyloxy)-2,2-dimethyl-hexanoic acid, calcium salt, are described in U.S. Pat. No. 5,648,387 and its divisionals U.S. Pat. Nos. 5,750,569; 5,756,544; and 5,783,600, and in PCT International Application Publication Nos. WO 96/30328; WO 01/55078.

Examples of substituted-alkyl compounds useful in the present invention include those of Formula II 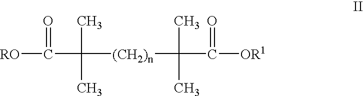
or a pharmaceutically acceptable salt thereof,
where n is 6, 7, 8, 9, or 10; and
R and R¹ are selected from the group consisting of hydrogen and C₁-C₈ alkyl.

Examples of compounds of Formula II include
2,2,9,9-tetramethyldecanedioic acid;
2,2,12,12-tetramethyltridecanedioic acid;
and pharmaceutically acceptable salts thereof.

Substituted-alkyl compounds of Formula II, and pharmaceutically acceptable salts thereof, are described in U.S. Pat. No. 3,773,946.

Examples of substituted-alkyl compounds useful in the present invention include those of Formula III 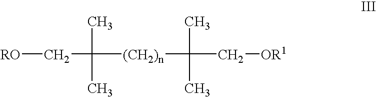
or a pharmaceutically acceptable salt thereof,
where n is 6, 7, 8, 9, or 10;
R and R¹ are selected from the group consisting of hydrogen, (C₁-C₁₂ alkyl)-C(═O)—, HO₂C(CH₂)m—CH₂—C(═O)—, phenyl-CH₂—C(H)(NH₂)—C(═O)—, and (HO)₂—P(═O)—; and
m is an integer of from 1 to 3; wherein alkyl is straight or branched.

Examples of compounds of Formula III include
2,2,9,9-tetramethyl-1,10-decanediol;
and a pharmaceutically acceptable salts thereof.

Substituted-alkyl compounds of Formula III, and pharmaceutically acceptable salts thereof, are described in U.S. Pat. No. 3,930,024.

Examples of substituted-aryl alkyl ether compounds useful in the present invention include those of Formula IV 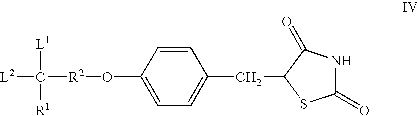
or a pharmaceutically acceptable salt thereof,
where
R¹ is C₁-C₁₀ alkyl, C₃-C₇ cycloalkyl, phenyl-(C₁-C₅ alkyl)-, phenyl, thienyl, furanyl, thiazolyl, pyridinyl, or R³R⁴N—;
R³ and R⁴ are the same or different C₁-C₄ alkyl, or R³ and R⁴ are combined to each other either directly, or as interrupted by a heteroatom selected from N, O, and S, with the nitrogen atom to which they are both bonded to form a 5- or 6-membered ring, wherein the 5- or 6-membered ring is piperidinyl, morpholinyl, pyrrolidinyl, or piperazinyl;
R² is a bond or —(CH₂)m—;
L¹ and L² are the same or different C₁-C₄ alkyl, or L¹ and L² are combined to each other to form —(CH₂)ₚ—;
p is an integer of from 2 to 6; and
when R¹ is C₃-C₇ cycloalkyl, phenyl-(C₁-C₅ alkyl)-, phenyl, thienyl, furanyl, thiazolyl, pyridinyl, or R³R⁴N—, L¹ and L² may further by hydrogen;
where the C₃-C₇ cycloalkyl, phenyl-(C₁-C₅ alkyl)-, phenyl, thienyl, furanyl, thiazolyl, pyridinyl, piperidinyl, morpholinyl, pyrrolidinyl, and piperazinyl groups may optionally have from 1 to 3 substituents independently selected from C₁-C₄ alkyl, (C₁-C₄ alkyl)-O—, F, Cl, Br, I, OH, and a methylenedioxy group of formula —O—(CH₂)m—O—, where the oxygen atoms of the methylenedioxy group are bonded to contiguous carbon atoms to form a ring of from 5 to 7 members; and
each m independently is an integer of from 1 to 3.

Examples of compounds of Formula IV include 5-[4-(1-methylcyclohexylmethyloxy)benzyl]thiazolidine-2,4-dione; a compound of any one of Examples 1 to 8, 10, and 11 of U.S. Pat. No. 4,287,200; any one of Compound Nos. 1 to 54 of Example 10 of U.S. Pat. No. 4,287,200; and any one of Compound Nos. 1 to 7 of Example 12 of U.S. Pat. No. 4,287,200; and pharmaceutically acceptable salts thereof.

Substituted aryl-alkyl ethers of Formula IV, and pharmaceutically acceptable salts thereof, are described in U.S. Pat. No. 4,287,200.

Examples of substituted dialkyl ether, substituted aryl-alkyl ether, substituted dialkyl thioether, substituted dialkyl ketone, or substituted-alkyl compounds useful in the present invention include those of Formula V 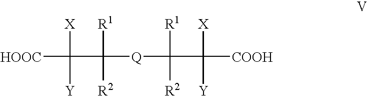
or a pharmaceutically acceptable salt thereof, or an in vivo hydrolyzable functional derivative selected from an ester, amide, or anhydride with (C₁-C₅ alkyl)-COOH;
where
R¹ and R² each independently represent an unsubstituted or substituted hydrocarbyl selected from C₁-C₆ alkyl optionally substituted by phenyl, OH, (C₁-C₆ alkyl)-O—, F, Cl, or Br, C₂-C₆ alkenyl, C₂-C₆ alkynyl, C₃-C₇ cycloalkyl, phenyl optionally substituted by OH, (C₁-C₆ alkyl)-O—, C₁-C₆ alkyl, F, Cl, or Br, or heterocyclyl;
X and Y each independently represent hydrogen, C₁-C₆ alkyl, F, Cl, Br, COOH, (C₁-C₆ alkyl)-O—C(═O)—, or (C₁-C₆ alkyl)-N(H)—C(═O)—, and further one of X and Y can also be (C₁-C₆ alkyl)-O—, HO, or NC—;

Q represents a diradical consisting of an alkylenyl diradical of from 8 to 14 carbon atoms or a heteroalkylenyl diradical of from 8 to 14 members having carbon atoms and a heteroatom selected from S, S(O), S(O)₂, N(H), N(C₁-C₆ alkyl), N(CH₂-phenyl), and O, where the alkylenyl or heteroalkylenyl may optionally be substituted by oxo (═O), F, Cl, Br, OH, or (C₁-C₆ alkyl)-O—, and where any from 1 to 4 contiguous atoms in the alkylenyl or heteroalkylenyl may comprise a C₃-C₇ cycloalkyl and where any from 2 to 4 contiguous atoms in the alkylenyl or heteroalkylenyl may comprise a phenyl.

Examples of compounds of Formula V include
2,3,3,14,14,15-hexamethyl-hexadecane-1,16-dioic acid;
2,15-di-carbamoyl-3,3,14,14-tetramethyl-hexadecane-1,16-dioic acid;
3,14-diethyl-3,14-dimethyl-hexadecane-1,16-dioic acid;
3,3,14,14-tetra-(2-propenyl)-hexadecane-1,16-dioic acid;
3,3,14,14-tetra-cyclohexyl-hexadecane-1,16-dioic acid;
2,15-dibromo-3,3,14,14-tetraphenyl-hexadecane-1,16-dioic acid;
1,2-cyclopropylidine-bis-(3,3-dimethyl-7-yl-heptanoic acid);
9,9-pentamethylene-3,3-15,15-tetramethyl-heptadecane-1,17-dioic acid;
1,2-cyclohexylidene-bis-(3,3-dimethyl-7-yl-heptanoic acid);
1,2-phenylene-(3,3-dimethyl-7-yl-heptanoic acid);
3,3,15,15-tetramethyl-9-thia-heptadecane-1,17-dioic acid;
9-oxa-3,3,15,15-tetramethyl-heptadecane-1,17-dioic acid;
9-aza-3,3,15,15-tetramethyl-heptadecane-1,17-dioic acid;
3,3,14,14-tetramethyl-6,11-dithiahexadecane-1,16-dioic acid;
2,15-difluoro-3,3,14,14-tetramethyl-hexadecane-1,16-dioic acid;
2,2,15,15-tetrafluoro-3,3,14,14-tetramethyl-hexadecane-1,16-dioic acid;
2,2,15,15-tetrachloro-3,3,14,14-tetramethyl-hexadecane-1,16-dioic acid;
3,3,14,14-tetrahydroxymethyl-hexadecane-1,16-dioic acid;
2,15-dichloro-3,14-di(chloromethyl)-3,14-dimethyl-hexadecane-1,16-dioic acid;
2,15-dichloro-3,3,14,14-tetra(chloromethyl)-hexadecane-1,16-dioic acid;
3,3,14,14-tetra-(4-hydroxyphenyl)-hexadecane-1,16-dioic acid;
3,3,14,14-tetra-(4-chlorophenyl)-hexadecane-1,16-dioic acid;
3,3,14,14-tetra-(4-methyl-phenyl)-hexadecane-1,16-dioic acid;
3,3,14,14-tetra-(4-methoxy-phenyl)-hexadecane-1,16-dioic acid;
and pharmaceutically acceptable salts thereof.
   Additional examples of compounds of Formula V include
1,1,14,14-tetra(ethoxycarbonyl)-2,2,13,13-tetramethyl-tetradecane;
1,1,16,16-tetra(ethoxycarbonyl)-2,2,15,15-tetramethyl-hexadecane;
1,1,12,12-tetra(ethoxycarbonyl)-2,2,11,11-tetramethyl-dodecane;
3,3,14,14-tetramethyl-hexadecane-1,16-dioic acid;
3,3,16,16-tetramethyl-octadecane-1,18-dioic acid;
3,3,12,12-tetramethyl-tetradecane-1,14-dioic acid;
1,14-di-(ethoxycarbonyl)-1,14-dicyano-2,2,13,13-tetramethyl-tetradecane;
2,15-dicyano-3,3,14,14-tetramethyl-hexadecane-1,16-dioic acid;
2,15-dibromo-3,3,14,14-tetramethyl-hexadecane-1,16-dioic acid;
2,3,3,14,14,15-hexamethyl-hexadecane-1,16-dioic acid;
1,14-diethoxycarbonyl-2,2,13,13-tetramethyl-tetradecane;
1,14-di-(ethoxycarbonyl)-1,14-dibromo-2,2,13,13-tetramethyl-tetradecane;
1,14-bis-carbamoyl-2,2,13,13-tetramethyl-tetradecane;
2,15-dichloro-3,3,14,14-tetramethylhexadecane-1,16-dioic acid;
2,15-dibromo-3,3,14,14-tetramethylhexadecane-1,16-dioic acid;
2,15-dihydroxy-3,3,14,14-tetramethylhexadecane-1,16-dioic acid;
1,14-di-(carbomethoxy)-1,14-dibromo-2,2,13,13-tetramethyltetradecane;
1,14-di-(carbomethoxy)-1,14-dichloro-2,2,13,13-tetramethyltetradecane;
2,15-dimethoxy-3,3,14,14-tetramethylhexadecane-1,16-dioic acid;
1,1,18,18-tetra(carboethoxy)-2,2,17,17-tetramethyloctadecane;
3,3,18,18-tetramethyleicosane-1,20-dioic acid;
3,3,14,14-tetramethyl-8-hexadecene-1,16-dioic acid;
3,3,14,14-tetraphenyl-6,11-diketohexadecane-1,16-dioic acid;
3,3,14,14-tetraphenylhexadecane-1,16-dioic acid;
1,4-phenylene-bis-[(1,1-dimethyl-but-4-yl)-dipropionic acid dimethyl ester];
1,4-phenylene-bis-[(1,1-dimethyl-but-4-yl)-dipropionic acid];
1,4-phenylene-bis(3,3-dimethyl-6-yl-5-hexenoic acid methyl ester);
1,3-phenylene-bis(3,3-dimethyl-6-yl-5-hexenoic acid methyl ester);
1,4-phenylene-bis(3,3-dimethyl-6-yl-hexanoic acid methyl ester);
1,3-phenylene-bis(3,3-dimethyl-6-yl-hexanoic acid methyl ester);
1,4-phenylene-bis(3,3-dimethyl-6-yl-hexanoic acid);
1,3-phenylene-bis(3,3-dimethyl-6-yl-hexanoic acid);
1,4-(cyclohexylidene-bis-(3,3-dimethyl-6-yl-hexanoic acid methyl ester);
1,3-(cyclohexylidene-bis-(3,3-dimethyl-6-yl-hexanoic acid methyl ester);
1,4-(cyclohexylidene-bis-(3,3-dimethyl-6-yl-hexanoic acid);
1,3-(cyclohexylidene-bis-(3,3-dimethyl-6-yl-hexanoic acid);
1,4-phenylene-bis(3,3-dimethyl-7-yl-5-heptenoic acid);
1,3-phenylene-bis(3,3-dimethyl-7-yl-5-heptenoic acid);
1,4-phenylene-bis(3,3-dimethyl-7-yl-heptanoic acid);
1,3-phenylene-bis(3,3-dimethyl-7-yl-heptanoic acid);
1,4-(cyclohexylidene-bis-(3,3-dimethyl-7-yl-heptanoic acid);
1,3-(cyclohexylidene-bis-(3,3-dimethyl-7-yl-heptanoic acid);
1,4-(cyclohexylidene-bis-(3,3-dimethyl-5-oxo-7-yl-heptanoic acid);
and pharmaceutically acceptable salts thereof.

Substituted dialkyl ether, substituted aryl-alkyl ether, substituted dialkyl thioether, substituted dialkyl ketone, or substituted-alkyl compounds of Formula V, and pharmaceutically acceptable salts thereof, are described in U.S. Pat. No. 4,689,344.

Examples of substituted dialkyl ether, substituted aryl-alkyl ether, substituted dialkyl thioether, substituted dialkyl ketone, or substituted-alkyl compounds useful in the present invention include those of Formula VI 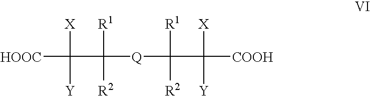
or a pharmaceutically acceptable salt thereof, or an in vivo hydrolyzable functional derivative selected from an ester, amide, or anhydride with (C₁-C₅ alkyl)-COOH;
where
R¹ and R² each independently represent an unsubstituted or substituted C₁-C₆ alkyl optionally substituted by OH, (C₁-C₆ alkyl)-O—, F, Cl, Br, or phenyl, wherein the phenyl optionally substituted one or more times by OH, (C₁-C₆ alkyl)-O—, C₁-C₆ alkyl, F, Cl, or Br, C₂-C₆ alkenyl, C₂-C₆ alkynyl, C₃-C₇ cycloalkyl, phenyl optionally substituted by OH, (C₁-C₆ alkyl)-O—, C₁-C₆ alkyl, F, Cl, or Br, or heterocycle;
X and Y each independently represent hydrogen, C₁-C₆ alkyl, (C₁-C₆ alkyl)-O—, HO, NC—, F, Cl, Br, COOH, (C₁-C₆ alkyl)-O—C(═O)—, or (C₁-C₆ alkyl)-N(H)—C(═O)—;
Q represents a diradical consisting of an alkylenyl diradical of from 8 to 14 carbon atoms or a heteroalkylenyl diradical of from 8 to 14 members having carbon atoms and a heteroatom selected from S, S(O), S(O)₂, N(H), N(C₁-C₆ alkyl), N(CH₂-phenyl), and O, where the alkylenyl or heteroalkylenyl may optionally be substituted by oxo (═O), F, Cl, Br, OH, or (C₁-C₆ alkyl)-O—, and where any from 1 to 4 contiguous atoms in the alkylenyl or heteroalkylenyl may comprise a C₃-C₇ cycloalkyl and where any from 2 to 4 contiguous atoms in the alkylenyl or heteroalkylenyl may comprise a phenyl.

Examples of compounds of Formula VI include
2,15-difluoro-3,3,14,14-tetramethyl-1,16-hexadecanedioic acid;
2,15-dichloro-3,3,14,14-tetramethyl-hexadecane-1,16-dioic acid diisopropyl ester;
2,2,15,15-tetrachloro-3,3,14,14-tetramethyl-hexadecane-1,16-dioic acid;
and pharmaceutically acceptable salts thereof.

Substituted dialkyl ether, substituted aryl-alkyl ether, substituted dialkyl thioether, substituted dialkyl ketone, or substituted-alkyl compounds of Formula VI, and pharmaceutically acceptable salts thereof, are described in U.S. Pat. No. 4,711,896.

Examples of substituted dialkyl ether, substituted aryl-alkyl ether, substituted dialkyl thioether, substituted dialkyl ketone, or substituted-alkyl compounds useful in the present invention include those of Formula VII 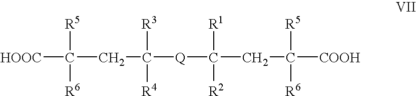
or a pharmaceutically acceptable salt thereof, or in vivo hydrolysable functional derivatives of the carboxylic groups thereof selected from C₁-C₆ alkyl ester, unsubstituted amide, C₁-C₆ alkyl amide, bis(C₁-C₆ alkyl) amide, anhydride with a C₁-C₆ carboxylic acid, and lactone formed by a dehydrative ring closure between a COOH group and any OH group of R⁵ or R⁶,
where
R¹, R², R³, and R⁴ each independently represents a hydrogen, an unsubstituted or substituted hydrocarbyl radical selected from C₁-C₆ alkyl, C₂-C₆ alkenyl, C₂-C₆ alkynyl, C₃-C₇ cycloalkyl, phenyl, and phenyl-(C₁-C₃ alkylenyl), or a heterocyclyl radical;
R⁵ and R⁶ independently represent hydrogen, hydroxyl, C₁-C₆ alkyl, chloro, bromo, cyano, nitro, C₁-C₆ alkoxy, or CF₃;
Q represents a diradical consisting of an unsubstituted or substituted linear chain of 2 to 14 carbon atoms, one or more of which may be replaced by heteroatoms selected from O, S, S(O), S(O)₂, N(H), N(C₁-C₆ alkyl), and N(CH₂phenyl);
where substituents are selected from oxo (═O), F, Cl, Br, OH, or (C₁-C₆ alkyl)-O—, and where any from 1 to 4 contiguous atoms in the in the linear chain may comprise a C₃-C₇ cycloalkyl and where any from 2 to 4 contiguous atoms in the linear chain may comprise a phenyl.

Additional examples of compounds of Formula VII include those where R¹, R², R³, R⁴, R⁵, and R⁶ are not each hydrogen.

Further examples of compounds of Formula VII include
4,4,11,11-tetramethyltetradecanedioic acid;
diethyl 4,4,13,13-tetramethylhexadeca-2,5,11,14-tetraenedionate;
4,4,13,13-tetramethylhexadecanedioic acid;
4,4,15,15-tetramethyloctadecanedioic acid;
2,2,15,15-tetramethylhexadecanedioic acid;
2,2,17,17-tetramethyloctadecanedioic acid;
and pharmaceutically acceptable salts thereof.

Substituted dialkyl ether, substituted aryl-alkyl ether, substituted dialkyl thioether, substituted dialkyl ketone, or substituted-alkyl compounds of Formula VII, and pharmaceutically acceptable salts thereof, are described in PCT International Patent Application Publication No. WO 98/30530.

Substituted dialkyl ether, substituted aryl-alkyl ether, substituted dialkyl thioether, substituted dialkyl ketone, or substituted-alkyl compounds, and pharmaceutically acceptable salts thereof, are described in U.S. patent application No. 10/205,939; U.S. Pat. Nos. 6,410,802; 6,459,003; and 6,506,799; in U.S. Patent Application Publication No. US 2003/0065195; and in PCT International Patent Application Publication No. WO 00/59855.

Substituted dialkyl ether, substituted aryl-alkyl ether, substituted dialkyl thioether, substituted dialkyl ketone, or substituted-alkyl compounds, and pharmaceutically acceptable salts thereof, are described in U.S. patent application Ser. No. 09/976,867; U.S. Patent Application Publication No. US 2003/0018013; and in PCT International Patent Application Publication No. WO 02/30863.

Substituted dialkyl thioethers are described in U.S. patent application Ser. Nos. 09/976,898; and 09/976,899; U.S. Patent Application Publication Nos. US 2002/0077316; and US 2003/0022865; and in PCT International Patent Application Publication Nos. WO 02/30882 and WO 02/30884.

Substituted dialkyl ketones are described in U.S. patent application Ser. No. 09/976,938; U.S. Patent Application Publication No. US 2003/0078239 and PCT International Patent Application Publication No. WO 02/30860.

It should be appreciated that the compounds utilized in an invention method, composition, or combination can generally be prepared by carrying out the procedures disclosed in those references above, herein incorporated by reference.

It should be appreciated that the compounds utilized in an invention method, composition, or combination are capable of further forming pharmaceutically acceptable salts, including, but not limited to, acid addition and/or base salts. The acid addition salts are formed from basic compounds, whereas the base addition salts are formed from acidic compounds. All of these forms are within the scope of the compounds useful in an invention method, composition, or combination.

Pharmaceutically acceptable acid addition salts of a substituted dialkyl ether, substituted aryl-alkyl ether, substituted dialkyl thioether, substituted dialkyl ketone, or substituted-alkyl compound include nontoxic salts derived from inorganic acids such as hydrochloric, nitric, phosphoric, sulfuric, hydrobromic, hydroiodic, hydrofluoric, phosphorous, and the like, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, malate, tartrate, methanesulfonate, and the like. Also contemplated are nontoxic salts of amino acids such as arginate and the like and gluconate, galacturonate (see, for example, Berge S. M. et al., “Pharmaceutical Salts,” J. of Pharma. Sci., 1977;66:1).

An acid addition salt of a substituted dialkyl ether, substituted aryl-alkyl ether, substituted dialkyl thioether, substituted dialkyl ketone, or substituted-alkyl compound is prepared by contacting the free base form of the compound with a sufficient amount of a desired acid to produce a nontoxic salt in the conventional manner. The free base form of the compound may be regenerated by contacting the acid addition salt so formed with a base, and isolating the free base form of the compound in the conventional manner. The free base forms of compounds differ from their respective acid addition salt forms somewhat in certain physical properties such as solubility, crystal structure, hygroscopicity, and the like, but otherwise free base forms of the compounds and their respective acid addition salt forms may be equally utilized in an invention method, composition, or combination.

A pharmaceutically acceptable base addition salt of a substituted dialkyl ether, substituted aryl-alkyl ether, substituted dialkyl thioether, substituted dialkyl ketone, or substituted-alkyl compound may be prepared by contacting the free acid form of the compound with a metal cation such as an alkali or alkaline earth metal cation, or an amine, especially an organic amine. Examples of suitable metal cations include sodium cation (Na⁺), potassium cation (K⁺), magnesium cation (Mg²⁺), calcium cation (Ca²⁺), and the like. Examples of suitable amines are N,N′-dibenzylethylenediamine, chloroprocaine, choline, diethanolamine, dicyclohexylamine, ethylenediamine, N-methylglucamine, and procaine (see, for example, Berge, supra., 1977).

A base addition salt of a substituted dialkyl ether, substituted aryl-alkyl ether, substituted dialkyl thioether, substituted dialkyl ketone, or substituted-alkyl compound may be prepared by contacting the free acid form of the compound with a sufficient amount of a desired base to produce the salt in the conventional manner. The free acid form of the compound may be regenerated by contacting the salt form so formed with an acid, and isolating the free acid of the compound in the conventional manner. The free acid forms of the compounds differ from their respective salt forms somewhat in certain physical properties such as solubility, crystal structure, hygroscopicity, and the like, but otherwise the salts may be utilized equally in an invention method, composition, or combination.

The compounds useful in an invention method, composition, or combination may exist in unsolvated forms as well as solvated forms, including hydrated forms. In general, the solvated forms, including hydrated forms, are equivalent to unsolvated forms. An invention method, composition, or combination may utilize any solvated form, including hydrated form, of the compound, as well as mixtures thereof.

The compounds useful in an invention method, composition, or combination may possess one or more chiral centers, and each center may exist in the R or S configuration. An invention method, composition, or combination may utilize any diastereomeric, enantiomeric, or epimeric form of a substituted dialkyl ether, substituted aryl-alkyl ether, substituted dialkyl thioether, substituted dialkyl ketone, or substituted-alkyl compound, or a pharmaceutically acceptable salt thereof, as well as mixtures thereof.

Certain compounds useful in an invention method, composition, or combination may exist as two or more tautomeric forms. Tautomeric forms of the substituted dialkyl ether, substituted aryl-alkyl ether, substituted dialkyl thioether, substituted dialkyl ketone, or substituted-alkyl compounds may interchange, for example, via enolization/de-enolization, 1,2-hydride, 1,3-hydride, or 1,4-hydride shifts, and the like. An invention method, composition, or combination may utilize any tautomeric form of the compound, as well as mixtures thereof.

Some compounds useful in an invention method, composition, or combination have alkenyl groups, which may exist as entgegen or zusammen conformations, in which case all geometric forms thereof, both entgegen and zusammen, cis and trans, and mixtures thereof, may be utilized in an invention method, composition, or combination.

Some compounds useful in an invention method, composition, or combination have cycloalkyl groups, which may be substituted at more than one carbon atom, in which case all geometric forms thereof, both cis and trans, and mixtures thereof, may be used in an invention method, composition, or combination.

Some compounds useful in an invention method, composition, or combination may exist as amorphous or crystalline solids, in which case all physical forms thereof, including clathrates thereof and mixtures thereof, may be used in an invention method, composition, or combination.

Invention methods, compositions, or combinations also utilize isotopically-labelled compounds useful in an invention method, composition, or combination, which are identical to those recited above, but for the fact that one or more atoms are replaced by an atom having an atomic mass or mass number different from the atomic mass or mass number usually found in nature. Examples of isotopes that can be incorporated into compounds utilized in an invention method, composition, or combination include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively. Compounds and pharmaceutically acceptable salts of said compounds that contain the aforementioned isotopes and/or other isotopes of other atoms may be utilized in an invention method, composition, or combination. Certain isotopically labelled compounds utilized in an invention method, composition, or combination, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are known for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be utilized in some circumstances. Isotopically labelled compounds of those described above in an invention method, composition, or combination can generally be prepared by carrying out the procedures incorporated by reference above and below, or procedures disclosed in the Schemes and/or in the Examples and Preparations, if any, disclosed herein, by substituting a readily available isotopically labelled reagent for a non-isotopically labelled reagent.

It should be appreciated that COX-2 is also known as prostaglandin synthase-2, prostaglandin PGH₂ synthase, and prostaglandin-H₂ synthase-2.

A selective inhibitor of COX-2 means compounds that inhibit COX-2 selectively versus COX-1 such that a ratio of IC₅₀ for a compound with COX-1 divided by a ratio of IC₅₀ for the compound with COX-2 is greater than, or equal to, 5, where the ratios are determined in one or more assays. All that is required to determine whether a compound is a selective COX-2 inhibitor is to assay a compound in one of a number of well know assays in the art.

Examples of selective inhibitors of COX-2 include
ABT-963;
Valdecoxib;
BMS-347070;
Celecoxib;
Tilacoxib;
The compound of formula (B) 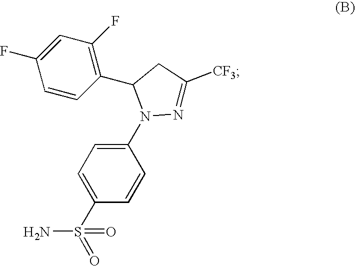
CS-502 [Chemical Abstracts Service Registry Number (“CAS Reg. No.”) 176429-82-6];
(6aR, 10aR)-3-(1,1-dimethylheptyl)-6a,7,10,10a-tetrahydro-1-hydroxy-6,6-dimethyl-6H-dibenzo[b,d]pyran-9-carboxylic acid (“CT-3”);
CV-247;
2(5H)-Furanone, 5,5-dimethyl-3-(1-methylethoxy)-4-[4-(methylsulfonyl)phenyl]-(“DFP”);
Carprofen (trade name RIMADYL® by Pfizer, Inc., New York, N.Y.);
Deracoxib (tradename DERAMAXX® by Novartis AG, Basel, Switzerland);
Etoricoxib (tradename ARCOXIA® by MERCK & CO., Inc., Whitehouse Station, N.J.);
GW-406381;
Tiracoxib;
Meloxicam;
Nimesulide;
2-(Acetyloxy)benzoic acid, 3-[(nitrooxy)methyl]phenyl ester (“NCX-4016”);
Lumiracoxib (tradename PREXIGE® by Novartis AG, Basel, Switzerland);
Parecoxib (trade name application pending for DYNASTAT® by G. D. Searle & Co., Skokie, Ill.);
P54 (CAS Reg. No. 130996-28-0);
Rofecoxib (tradename VIOXX® by MERCK & CO., Inc., Whitehouse Station, N.J.);
RevlMiD;
2,6-Bis(1,1-dimethylethyl)-4-[(E)-(2-ethyl-1,1-dioxo-5-isothiazolidinylidene)methyl]phenol (“S-2474”);
5(R)-Thio-6-sulfonamide-3(2H)-benzofuranone (“SVT-2016”); and
N-[3-(Formylamino)-4-oxo-6-phenoxy-4H-1-benzopyran-7-yl]-methanesulfonamide (“T-614”); and
pharmaceutically acceptable salts thereof.

The term “celecoxib” means the compound named 4-(5-(4-methylphenyl)-3-(trifluoromethyl)-1H-pyrazol-1-yl)-benzenesulfonamide. Celecoxib is a selective cyclooxygenase-2 (“COX-2”) inhibitor currently approved by the FDA for the treatment of osteoarthritis, rheumatoid arthritis, and Polyposis-familial adenomatus. Celecoxib is marketed under the tradename “CELEBREX®”. Celecoxib is currently in clinical trials for the treatment of bladder cancer, chemopreventative-lung cancer, and post-operative pain, and is registered for the treatment of dysmenorrhea. Celecoxib has the structure drawn below:

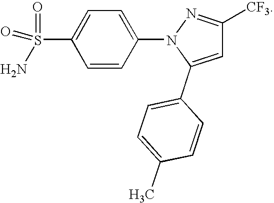

The term “valdecoxib” means the compound named 4-(5-methyl-3-phenyl-4-isoxazolyl)-benzenesulfonamide, which is described in U.S. Pat. Nos. 5,633,272; 5,859,257; and 5,985,902, which are hereby incorporated by reference herein. Valdecoxib has been approved by the FDA for treating osteoarthritis, rheumatoid arthritis, dysmenorrhea, and general pain, and is marketed under the tradename “BEXTRA®”. Valdecoxib is in clinical trials for the treatment of migraine. Valdecoxib has the structure drawn below:

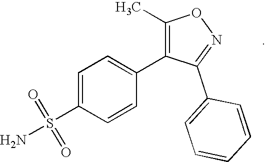

It should also be appreciated that carprofen and deracoxib are each indicated for the treatment of arthritis in an animal, especially a dog.

As previously mentioned, the co-administration of a substituted dialkyl ether, substituted aryl-alkyl ether, substituted dialkyl thioether, substituted dialkyl ketone, or substituted-alkyl compound, or a pharmaceutically acceptable salt thereof, and a selective COX-2 inhibitor, or a pharmaceutically acceptable salt thereof, is effective in the treatment and prevention of inflammation and inflammation-associated disorders including, for example, osteoarthritis, rheumatoid arthritis, osteoarthritic joint pain, rheumatoid arthritic joint, joint pain, inflammatory pain, acute pain, chronic pain, and cartilage damage.

An active compound having an anti-inflammatory, an analgesic, anti-arthritic, or a cartilage damage inhibiting effect, or any combination of these effects, may be readily identified by one of ordinary skill in the pharmaceutical or medical arts by assaying the substituted dialkyl ether, substituted aryl-alkyl ether, substituted dialkyl thioether, substituted dialkyl ketone, or substituted-alkyl compound, and selective COX-2 inhibitor in any number of well known assays for measuring determining the substituted dialkyl ether, substituted aryl-alkyl ether, substituted dialkyl thioether, substituted dialkyl ketone, or substituted-alkyl compound, and selective COX-2 inhibitor compound's effects on cartilage damage, arthritis, inflammation, or pain. These assays include in vitro assays that utilize cartilage samples and in vivo assays in whole animals that measure cartilage degradation, inhibition of inflammation, or pain alleviation.

For example with regard to assaying cartilage damage in vitro, an amount of an active compound or control vehicle may be administered with a cartilage damaging agent to cartilage, and the cartilage damage inhibiting effects in both tests studied by gross examination or histopathologic examination of the cartilage, or by measurement of biological markers of cartilage damage such as, for example, proteoglycan content or hydroxyproline content. Further, in vivo assays to assay cartilage damage may be performed as follows: an amount of an active compound or control vehicle may be administered with a cartilage damaging agent to an animal, and the effects of the substituted dialkyl ether, substituted aryl-alkyl ether, substituted dialkyl thioether, substituted dialkyl ketone, or substituted-alkyl compound, and selective COX-2 inhibitor being assayed on cartilage in the animal may be evaluated by gross examination or histopathologic examination of the cartilage, by observation of the effects in an acute model on functional limitations of the affected joint that result from cartilage damage, or by measurement of biological markers of cartilage damage such as, for example, proteoglycan content or hydroxyproline content.

Several methods of identifying an active compound with cartilage damage inhibiting properties are described below. The amount to be administered in an assay is dependent upon the particular assay employed, but in any event is not higher than the well-known maximum amount of a compound that the particular assay can effectively accommodate.

Similarly, substituted dialkyl ether, substituted aryl-alkyl ether, substituted dialkyl thioether, substituted dialkyl ketone, or substituted-alkyl compound, and selective COX-2 inhibitors having pain-alleviating properties may be identified using any one of a number of in vivo animal models of pain. A number of in vivo animal models of joint pain are known in the art, and a model of endothelin-1 mediated pain is described by Piovezan, Anna P., et al., British Journal of Pharmacology, 2000;129:961-968, which is incorporated herein by reference.

Still similarly, substituted dialkyl ether, substituted aryl-alkyl ether, substituted dialkyl thioether, substituted dialkyl ketone, or substituted-alkyl compound, and selective COX-2 inhibitors having anti-inflammatory properties may be identified using any one of a number of in vivo animal models of inflammation. For example, for an example of inflammation models, see U.S. Pat. No. 6,329,429, which is incorporated herein by reference.

Still similarly, substituted dialkyl ether, substituted aryl-alkyl ether, substituted dialkyl thioether, substituted dialkyl ketone, or substituted-alkyl compound, and selective COX-2 inhibitors having anti-arthritic properties may be identified using any one of a number of in vivo animal models of arthritis. For example, for an example of arthritis models, see also U.S. Pat. No. 6,329,429.

The combinations of substituted dialkyl ether, substituted aryl-alkyl ether, substituted dialkyl thioether, substituted dialkyl ketone, or substituted-alkyl compound, and selective COX-2 inhibitor disclosed herein are also useful in the treatment and prevention of inflammation and inflammation-associated disorders mediated by proinflammatory cytokines such as interleukin-6 (IL-6), interleukin-1β (IL-1β), interleukin-6 soluble receptor (IL-6sR), or a combination of IL-6, IL-6sR and IL-1β.

The combinations of substituted dialkyl ether, substituted aryl-alkyl ether, substituted dialkyl thioether, substituted dialkyl ketone, or substituted-alkyl compound, and selective COX-2 inhibitor disclosed herein are also useful in the treatment and prevention of inflammation and inflammation-associated disorders mediated by proinflammatory cytokine induced CRP production. CRP protein concentrations can be determined using a CRP ELISA Kit (Alpha Diagnostic International, Inc. San Antonio, Tex. USA).

The compositions disclosed herein can also be used in combination with existing therapeutic agents for the treatment of osteoarthritis or rheumatoid arthritis, the alleviation of pain, or other inflammation or inflammation-associated ailments. Suitable agents to be used in combination include standard non-steroidal anti-inflammatory agents (hereinafter NSAID's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, and carprofen analgesics and intraarticular therapies such as corticosteroids and hyaluronic acids such as hyalgan and synvisc.

Another aspect of the invention relates to a method of, and a pharmaceutical composition for, treating or preventing inflammation or inflammation-associated diseases comprising administering a combination of dialkyl ether, substituted aryl-alkyl ether, substituted dialkyl thioether, substituted dialkyl ketone, or substituted-alkyl compound and a selective COX-2 inhibitor to a mammal with one or more other therapeutically active agents under the following conditions:
A.) where a joint has become seriously inflamed as well as infected at the same time by bacteria, fungi, protozoa and/or virus, said inhibitory combination is administered in combination with one or more antibiotic, antifungal, antiprotozoal and/or antiviral therapeutic agents;
B.) where a multi-fold treatment of pain and inflammation is desired, said inhibitory combination is administered in combination with inhibitors of other mediators of inflammation, comprising one or more members independently selected from the group consisting essentially of:
(1) NSAIDs;
(2) H₁-receptor antagonists;
(3) kinin-B₁- and B₂-receptor antagonists;
(4) prostaglandin inhibitors selected from the group consisting of PGD-, PGF- PGI₂- and PGE-receptor antagonists;
(5) thromboxane A₂ (TXA₂-) inhibitors;
(6) 5-, 12- and 15-lipoxygenase inhibitors;
(7) leukotriene LTC₄-, LTD₄/LTE₄- and LTB₄-inhibitors;
(8) PAF-receptor antagonists;
(9) gold in the form of an aurothio group together with one or more hydrophilic groups;
(10) immunosuppressive agents selected from the group consisting of cyclosporine, azathioprine and methotrexate;
(11) anti-inflammatory glucocorticoids;
(12) penicillamine;
(13) hydroxychloroquine;
(14) anti-gout agents including colchicine; xanthine oxidase inhibitors including allopurinol; and uricosuric agents selected from probenecid, sulfinpyrazone and benzbromarone;
C. where older mammals are being treated for disease conditions, syndromes and symptoms found in geriatric mammals, said inhibitory combination is administered in combination with one or more members independently selected from the group consisting essentially of:
(1) cognitive therapeutics to counteract memory loss and impairment;
(2) anti-hypertensives and other cardiovascular drugs intended to offset the consequences of atherosclerosis, hypertension, myocardial ischemia, angina, congestive heart failure and myocardial infarction, selected from the group consisting of:
a. diuretics;
b. vasodilators;
c. β-adrenergic receptor antagonists;
d. angiotensin-II converting enzyme inhibitors (ACE-inhibitors), alone or optionally together with neutral endopeptidase inhibitors;
e. angiotensin II receptor antagonists;
f. renin inhibitors;
g. calcium channel blockers;
h. sympatholytic agents;
i. α₂-adrenergic agonists;
j. α-adrenergic receptor antagonists; and
k. HMG-CoA-reductase inhibitors (anti-hypercholesterolemics);
(3) antineoplastic agents selected from:
a. antimitotic drugs selected from:
i. vinca alkaloids selected from:
[1] vinblastine and
[2] vincristine;
(4) growth hormone secretagogues;
(5) strong analgesics;
(6) local and systemic anesthetics; and
(7) H₂-receptor antagonists, proton pump inhibitors and other gastroprotective agents.

The compositions disclosed herein may be administered in combination with inhibitors of other mediators of inflammation, comprising one or more members selected from the group consisting essentially of the classes of such inhibitors and examples thereof which include, matrix metalloproteinase inhibitors, aggrecanase inhibitors, TACE inhibitors, leucotriene receptor antagonists, IL-1 processing and release inhibitors, ILra, H₁-receptor antagonists; kinin-B₁- and B₂-receptor antagonists; prostaglandin inhibitors such as PGD-, PGF- PGI₂- and PGE-receptor antagonists; thromboxane A₂ (TXA2-) inhibitors; 5- and 12-lipoxygenase inhibitors; leukotriene LTC₄-, LTD₄/LTE₄- and LTB₄-inhibitors; PAF-receptor antagonists; MEK inhibitors; IKK inhibitors; MKK inhibitors; gold in the form of an aurothio group together with various hydrophilic groups; immunosuppressive agents, e.g., cyclosporine, azathioprine and methotrexate; anti-inflammatory glucocorticoids; penicillamine; hydroxychloroquine; anti-gout agents, e.g., colchicine, xanthine oxidase inhibitors, e.g., allopurinol and uricosuric agents, e.g., probenecid, sulfinpyrazone and benzbromarone.

The compositions disclosed herein may also be used in combination with anticancer agents such as endostatin and angiostatin or cytotoxic drugs such as adriamycin, daunomycin, cis-platinum, etoposide, taxol, taxotere and alkaloids, such as vincristine and antimetabolites such as methotrexate.

The compositions disclosed herein may also be used in combination with anti-hypertensives and other cardiovascular drugs intended to offset the consequences of atherosclerosis, including hypertension, myocardial ischemia including angina, congestive heart failure and myocardial infarction, selected from vasodilators such as hydralazine, β-adrenergic receptor antagonists such as propranolol, calcium channel blockers such as nifedipine, α₂-adrenergic agonists such as clonidine, α-adrenergic receptor antagonists such as prazosin and HMG-CoA-reductase inhibitors (anti-hypercholesterolemics) such as lovastatin or atorvastatin.

The compositions disclosed herein may also be administered in combination with one or more antibiotic, antifungal, antiprotozoal, antiviral or similar therapeutic agents.

The compositions disclosed herein may also be used in combination with CNS agents such as antidepressants (such as sertraline), anti-Parkinsonian drugs (such as L-dopa, requip, mirapex, MAOB inhibitors such as selegine and rasagiline, comP inhibitors such as Tasmar, A-2 inhibitors, dopamine reuptake inhibitors, NMDA antagonists, nicotine agonists, dopamine agonists and inhibitors of neuronal nitric oxide synthase) and anti-Alzheimer's drugs such as donepezil, tacrine, COX-2 inhibitors, propentofylline or metryfonate.

The compositions disclosed herein may also be used in combination with osteoporosis agents such as roloxifene, lasofoxifene, droloxifene or fosomax and immunosuppressant agents such as FK-506 and rapamycin.

Other inflammation-associated diseases which are treatable by co-administration of a substituted dialkyl ether, substituted aryl-alkyl ether, substituted dialkyl thioether, substituted dialkyl ketone, or substituted-alkyl compound, and a selective COX-2 inhibitor include: fever (including rheumatic fever and fever associated with influenza and other viral infections), common cold, dysmenorrhea, menstrual cramps, inflammatory bowel disease, Crohn's disease, emphysema, acute respiratory distress syndrome, asthma, bronchitis, chronic obstructive pulmonary disease, Alzheimer's disease, organ transplant toxicity, cachexia, allergic reactions, allergic contact hypersensitivity, cancer (such as solid tumor cancer including colon cancer, breast cancer, lung cancer and prostrate cancer; hematopoietic malignancies including leukemias and lymphomas; Hodgkin's disease; aplastic anemia, skin cancer and familiar adenomatous polyposis), tissue ulceration, peptic ulcers, gastritis, regional enteritis, ulcerative colitis, diverticulitis, recurrent gastrointestinal lesion, gastrointestinal bleeding, coagulation, anemia, synovitis, gout, ankylosing spondylitis, restenosis, periodontal disease, epidermolysis bullosa, osteoporosis, loosening of artificial joint implants, atherosclerosis (including atherosclerotic plaque rupture), aortic aneurysm (including abdominal aortic aneurysm and brain aortic aneurysm), periarteritis nodosa, congestive heart failure, myocardial infarction, stroke, cerebral ischemia, head trauma, spinal cord injury, neuralgia, neuro-degenerative disorders (acute and chronic), autoimmune disorders, Huntington's disease, Parkinson's disease, migraine, depression, peripheral neuropathy, pain (including low back and neck pain, headache and toothache), gingivitis, cerebral amyloid angiopathy, nootropic or cognition enhancement, amyotrophic lateral sclerosis, multiple sclerosis, ocular angiogenesis, corneal injury, macular degeneration, conjunctivitis, abnormal wound healing, muscle orjoint sprains or strains, tendonitis, skin disorders (such as psoriasis, eczema, scleroderma and dermatitis), myasthenia gravis, polymyositis, myositis, bursitis, burns, diabetes (including types I and II diabetes, diabetic retinopathy, neuropathy and nephropathy), tumor invasion, tumor growth, tumor metastasis, corneal scarring, scleritis, immunodeficiency diseases (such as AIDS in humans and FLV, FIV in cats), sepsis, premature labor, hypoprothrombinemia, hemophilia, thyroiditis, sarcoidosis, Behcet's syndrome, hypersensitivity, kidney disease, Rickettsial infections (such as Lyme disease, Erlichiosis), Protozoan diseases (such as malaria, giardia, coccidia), reproductive disorders (for example in livestock), epilepsy, convulsions, and septic shock.

In determining what constitutes a therapeutically effective amount of a substituted dialkyl ether, substituted aryl-alkyl ether, substituted dialkyl thioether, substituted dialkyl ketone, or substituted-alkyl compound, or a pharmaceutically acceptable salt thereof, and selective COX-2 inhibitor, or a pharmaceutically acceptable salt thereof, for alleviating pain, preventing or treating osteoarthritis, preventing or treating rheumatoid arthritis, improving joint function, preventing or inhibiting cartilage damage, inhibiting proinflammatory cytokine induced CRP production, or treating or preventing inflammation and inflammation-associated disorders mediated by proinflammatory cytokines according to the invention method, a number of factors will generally be considered by the medical practitioner or veterinarian in view of the experience of the medical practitioner or veterinarian, published clinical studies, the subject's (ie, mammal's) age, sex, weight and general condition, as well as the type and extent of the disease, disorder or condition being treated, and the use of other medications, if any, by the subject. Such amounts will generally be from about 0.1 mg/kg to about 300 mg/kg of subject body weight. Typical doses will be from about 10 to about 5000 mg/day for an adult subject of normal weight. In a clinical setting, regulatory agencies such as, for example, the FDA in the United States may require a particular therapeutically effective amount.

As such, the administered dose may fall within the ranges or amounts recited above, or may vary outside, (for example, either below or above), those ranges depending upon the requirements of the individual subject, the severity of the condition being treated, and the particular therapeutic formulation being employed. Determination of a proper dose for a particular situation is within the skill of the medical or veterinary arts. Generally, treatment may be initiated using smaller dosages of an active compound useful in the invention method, or a pharmaceutically acceptable salt thereof, or a combination of the same with another therapeutic agent, that are less than optimum for a particular subject. Thereafter, the dosage can be increased by small increments until the optimum effect under the circumstance is reached. For convenience, the total daily dosage may be divided and administered in portions during the day, if desired.

The pharmaceutical compositions disclosed herein which include a combination of dialkyl ether, substituted aryl-alkyl ether, substituted dialkyl thioether, substituted dialkyl ketone, or substituted-alkyl compound and a selective COX-2 inhibitor can be formulated and administered to a mammalian host, such as a human patient in a variety of forms adapted to the chosen route of administration, i.e., orally or parenterally, by intravenous, intramuscular, or subcutaneous routes.

Thus, the combination of dialkyl ether, substituted aryl-alkyl ether, substituted dialkyl thioether, substituted dialkyl ketone, or substituted-alkyl compound and a selective COX-2 inhibitor may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. The amount of active compounds in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Any material used in preparing any unit dosage form should be pharmaceutically acceptable and substantially non-toxic in the amounts employed. In addition, the combination of dialkyl ether, substituted aryl-alkyl ether, substituted dialkyl thioether, substituted dialkyl ketone, or substituted-alkyl compound and a selective COX-2 inhibitor may be incorporated into sustained-release preparations and devices.

The combination of dialkyl ether, substituted aryl-alkyl ether, substituted dialkyl thioether, substituted dialkyl ketone, or substituted-alkyl compound and a selective COX-2 inhibitor may also be administered intravenously or intraperitoneally by infusion or injection. Solutions of the active compounds or their salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the combination of dialkyl ether, substituted aryl-alkyl ether, substituted dialkyl thioether, substituted dialkyl ketone, or substituted-alkyl compound and a selective COX-2 inhibitor which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the combination of dialkyl ether, substituted aryl-alkyl ether, substituted dialkyl thioether, substituted dialkyl ketone, or substituted-alkyl compound and a selective COX-2 inhibitor in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

The percentage of the active ingredients in the foregoing compositions can be varied within wide limits, but for practical purposes may be present in a concentration of at least 10% in a solid composition and at least 2% in a primary liquid composition, both up to about 95%.

Typical routes of administration of the active compounds useful in the invention method, or a pharmaceutically acceptable salt thereof, are oral or parenteral. For example, a useful intravenous dose is between 5 and 50 mg, and a useful oral dosage is between 20 and 800 mg. The dosage is within the dosing range used in treatment of inflammation or inflammation-associated diseases, such as those resulting in cartilage damage, loss of joint function, or pain for example rheumatoid arthritis and osteoarthritis, or as would be determined by the physician according to the needs of the patient as described above.

Useful dosages of the combination of dialkyl ether, substituted aryl-alkyl ether, substituted dialkyl thioether, substituted dialkyl ketone, or substituted-alkyl compound and a selective COX-2 inhibitor can be determined by comparing their in vitro activity, and in vivo activity in animal models. The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The following examples illustrate the various embodiments of the present invention. Those skilled in the art will recognize many variations that are within the spirit of the present invention and scope of the claims.

### BIOLOGICAL ASSAYS

The ability of the co-administration of a substituted dialkyl ether, substituted aryl-alkyl ether, substituted dialkyl thioether, substituted dialkyl ketone, or substituted-alkyl compound, or a pharmaceutically acceptable salt thereof, and a selective COX-2 inhibitor, or a pharmaceutically acceptable salt thereof to treat or prevent inflammation or an inflammation-associated disorder is demonstrated using pharmacological models that are well known to the art, for example, using models such as the tests described below.

Monosodium Iodoacetate (“MIA”)-induced Osteoarthritis:

Male Wistar rats (175-200 g) were housed in solid bottom isolator cages, 2-4 rats per cage, with corncob bedding on a 12 hour:12 hour light:dark cycle. Animals were fed standard rat chow with water available ad libitum.

The rats were anesthetized with 5% volume/volume (“v/v”) isoflurane gas and maintained with 2% v/v isoflurane gas. The anesthetized rats were given a single intra-articular injection of 1 mg of MIA through the infrapatellar ligament of the right knee. MIA was dissolved in physiologic saline and administered in a volume of 50 μL. The contralateral control knee was injected with 50 μL of physiologic saline. Administration of isoflurane gas was discontinued, and the rats became fully conscious about 5 minutes later.

Shifts in hind paw weight distribution from the right to the left hind paws supporting the right (arthritic) and the left (contralateral control) hind leg knee joints were utilized as an index of joint pain and as a measure of compound efficacy. An incapacitance tester (Model 2KG, Linton Instrumentation, UK) was employed for determination of hind paw weight distribution. Each data point is the mean of three readings of 5 seconds duration.

A solution was prepared by dissolving 6-(5-carboxy-5-methyl-hexyloxy)-2,2-dimethyl-hexanoic acid, calcium salt and rofecoxib (100 mg) in hydroxypropylmethylcellulose (“HPMC”) vehicle (0.05% HPMC+0.2% Tween 80; the amount of the 6-(5-carboxy-5-methyl-hexyloxy)-2,2-dimethyl-hexanoic acid used was adjusted based on the percent of free acid.

The acute dosing paradigm used herein relates to osteoarthritis signs such as mobility and joint function and osteoarthritis symptoms such as joint pain. In this dosing paradigm, changes in hind paw weight distribution were determined early on Day 14 post MIA injection, as described previously, to establish a baseline pain reading. Rats were then given a 10 mg/kg, dose of 6-(5-carboxy-5-methyl-hexyloxy)-2,2-dimethyl-hexanoic acid, calcium salt, rofecoxib (3 mg/kg), or the combination of the two, respectively, via oral gavage (PO). Changes in hind paw weight distribution were determined 2, 4 and 6 hours post-compound administration.

Results for joint pain alleviation following acute administration:

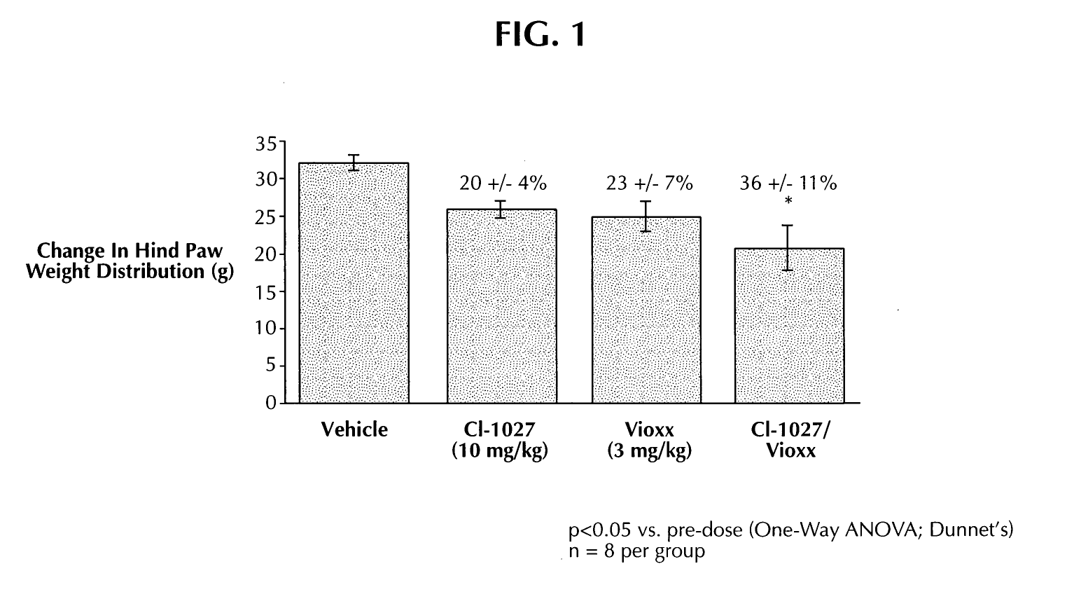

6-(5-carboxy-5-methyl-hexyloxy)-2,2-dimethyl-hexanoic acid, calcium salt and rofecoxib was tested in the rat MIA model in an acute dosing paradigm as described previously. MIA was injected into the right knee and saline into the left knee of all rats on Day 0. On Day 14 the rats were assessed on an incapacitance tester and then given 6-(5-carboxy-5-methyl-hexyloxy)-2,2-dimethyl-hexanoic acid, calcium salt (10 mg/kg, PO) and rofecoxib (3 mg/kg, PO). Two hours later, the rats were re-assessed. The results, graphically displayed in FIG. 1, show the change in rat hind paw weight distribution, (expressed in grams) in rats with monosodium iodoacetate (“MIA”)-induced knee joint arthritis. As shown in FIG. 1, administration of 6-(5-carboxy-5-methyl-hexyloxy)-2,2-dimethyl-hexanoic acid, calcium salt and rofecoxib altered the shift in weight bearing potential (joint pain) in arthritic rats at 2 hours post administration of 6-(5-carboxy-5-methyl-hexyloxy)-2,2-dimethyl-hexanoic acid, calcium salt and rofecoxib in a statistically significant manner compared to pre-dose measurements as well as compared to 6-(5-carboxy-5-methyl-hexyloxy)-2,2-dimethyl-hexanoic acid, calcium salt or rofecoxib alone.

Statistically significant differences were determined by one-way ANOVA followed by Dunnett's multiple comparisons procedure. Data are expressed as mean±SEM. N=8 rats per group.

### EXAMPLES

### Formulation Example 1

Tablet Formulation:

| Ingredient | Amount (mg) |
|---|---|
| 6-(5-carboxy-5-methyl-hexyloxy)-2,2-dimethyl- | 25 |
| hexanoic acid, calcium salt | |
| Rofecoxib | 20 |
| Lactose | 50 |
| Cornstarch (for mix) | 10 |
| Cornstarch (paste) | 10 |
| Magnesium stearate (1%) | 5 |
| Total | 120 |

6-(5-carboxy-5-methyl-hexyloxy)-2,2-dimethyl-hexanoic acid, calcium salt, rofecoxib, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for treatment of one of the above-listed diseases, including rheumatoid arthritis.

### Formulation Example 2

Coated Tablets:

The tablets of Formulation Example 9 are coated in a customary manner with a coating of sucrose, potato starch, talc, tragacanth, and colorant.

### Formulation Example 3

Injection Vials:

The pH of a solution of 250 g of rofecoxib, 500 g of 6-(5-carboxy-5-methyl-hexyloxy)-2,2-dimethyl-hexanoic acid, calcium salt, and 5 g of disodium hydrogen phosphate is adjusted to pH 6.5 in 3 L of double-distilled water using 2 M hydrochloric acid. The solution is sterile filtered, and the filtrate is filled into injection vials, lyophilized under sterile conditions, and aseptically sealed. Each injection vial contains 12.5 mg of rofecoxib and 25 mg of 6-(5-carboxy-5-methyl-hexyloxy)-2,2-dimethyl-hexanoic acid, calcium salt.

### Formulation Example 4

Suppositories:

A mixture of 50 g of rofecoxib, 25 g of 6-(5-carboxy-5-methyl-hexyloxy)-2,2-dimethyl-hexanoic acid, calcium salt, 100 g of soya lecithin, and 1400 g of cocoa butter is fused, poured into molds, and allowed to cool. Each suppository contains 50 mg of rofecoxib and 25 mg of 6-(5-carboxy-5-methyl-hexyloxy)-2,2-dimethyl-hexanoic acid, calcium salt.

### Formulation Example 5

Solution:

A solution is prepared from 0.5 g of rofecoxib, 1 g of 6-(5-carboxy-5-methyl-hexyloxy)-2,2-dimethyl-hexanoic acid, calcium salt, 9.38 g of NaH₂PO₄.12H₂O, 28.48 g of Na₂HPO₄.12H₂O, and 0.1 g benzalkonium chloride in 940 mL of double-distilled water. The pH of the solution is adjusted to pH 6.8 using 2 M hydrochloric acid. The solution is diluted to 1.0 L with double-distilled water, and sterilized by irradiation. A 25 mL volume of the solution contains 12.5 mg of rofecoxib and 25 mg of 6-(5-carboxy-5-methyl-hexyloxy)-2,2-dimethyl-hexanoic acid, calcium salt.

### Formulation Example 6

Ointment:

100 mg of rofecoxib, 500 mg of 6-(5-carboxy-5-methyl-hexyloxy)-2,2-dimethyl-hexanoic acid, calcium salt is mixed with 99.4 g of petroleum jelly under aseptic conditions. A 5 g portion of the ointment contains 5 mg of rofecoxib and 25 mg of 6-(5-carboxy-5-methyl-hexyloxy)-2,2-dimethyl-hexanoic acid, calcium salt.

### Formulation Example 7

Capsules:

2 kg of rofecoxib and 20 kg of 6-(5-carboxy-5-methyl-hexyloxy)-2,2-dimethyl-hexanoic acid, calcium salt are filled into hard gelatin capsules in a customary manner such that each capsule contains 25 mg of rofecoxib and 250 mg of 6-(5-carboxy-5-methyl-hexyloxy)-2,2-dimethyl-hexanoic acid, calcium salt.

### Formulation Example 8

Ampoules:

A solution of 2.5 kg of rofecoxib and 2.5 kg of 6-(5-carboxy-5-methyl-hexyloxy)-2,2-dimethyl-hexanoic acid, calcium salt is dissolved in 60 L of double-distilled water. The solution is sterile filtered, and the filtrate is filled into ampoules. The ampoules are lyophilized under sterile conditions and aseptically sealed. Each ampoule contains 25 mg each of rofecoxib and 6-(5-carboxy-5-methyl-hexyloxy)-2,2-dimethyl-hexanoic acid, calcium salt.

### Formulation Example 9

Tablet Formulation of 6-(5-carboxy-5-methyl-hexyloxy)-2,
2-dimethyl-hexanoic acid, calcium salt:

| Ingredient | Amount (mg) |
|---|---|
| 6-(5-carboxy-5-methyl-hexyloxy)-2, | 25 |
| 2-dimethyl-hexanoic acid, calcium salt | |
| Lactose | 50 |
| Cornstarch (for mix) | 10 |
| Cornstarch (paste) | 10 |
| Magnesium stearate (1%) | 5 |
| Total | 100 |

6-(5-carboxy-5-methyl-hexyloxy)-2,2-dimethyl-hexanoic acid, calcium salt, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet.

Injection Vial Formulation of Rofecoxib:

The pH of a solution of 500 g of rofecoxib and 5 g of disodium hydrogen phosphate is adjusted to pH 6.5 in 3 L of double-distilled water using 2 M hydrochloric acid. The solution is sterile filtered, and the filtrate is filled into injection vials, lyophilized under sterile conditions, and aseptically sealed. Each injection vial contains 25 mg of rofecoxib.

Such tablets containing 6-(5-carboxy-5-methyl-hexyloxy)-2,2-dimethyl-hexanoic acid, calcium salt can be administered to a human from one to four times a day for treatment of the above-listed diseases, and the injection solutions containing rofecoxib can be administered to a human 1 or 2 times per day, wherein the administration by injection is optionally simultaneous with administration of the tablets or at different times, for the treatment of one of the above-listed diseases, including rheumatoid arthritis.

### Formulation Example 10

Coated Tablets Containing 6-(5-carboxy-5-methyl-hexyloxy)-2,2-dimethyl-hexanoic Acid, Calcium Salt:

The tablets of Formulation Example 9 are coated in a customary manner with a coating of sucrose, potato starch, talc, tragacanth, and colorant.

Capsules Containing Rofecoxib:

2 kg of rofecoxib are filled into hard gelatin capsules in a customary manner such that each capsule contains 25 mg of rofecoxib.

Such coated tablets containing 6-(5-carboxy-5-methyl-hexyloxy)-2,2-dimethyl-hexanoic acid, calcium salt can be administered to a human from one to four times a day for treatment of the above-listed diseases, and the capsules containing rofecoxib can be administered to a human 1 or 2 times per day, wherein the administration of the capsules is optionally simultaneous with administration of the tablets or at different times, for the treatment of one of the above-listed diseases.

While the invention has been described and illustrated above with reference to certain particular aspects and embodiments thereof, those skilled in the art will appreciate that various adaptations, changes, modifications, substitutions, deletions, or additions of procedures and protocols may be made without departing from the spirit and scope of the invention.

All patents, patent applications, and publications, including patent application publications, cited herein are hereby incorporated by reference in their entirety for all purposes.

1. A method of treating or preventing inflammation or an inflammation-associated disorder selected from the group consisting of atherosclerosis, congestive heart failure, myocardial infarction, and stroke in a subject, said method comprising co-administering to the subject having or susceptible to such inflammation or inflammation-associated disorder, a therapeutically-effective amount of a COX-2 inhibitor or a pharmaceutically acceptable salt thereof and a therapeutically-effective amount of a substituted dialkyl ether compound of Formula I 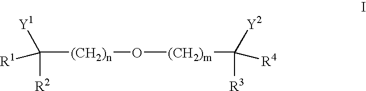
or a pharmaceutically acceptable salt thereof,
wherein:
n and m independently are integers of from 2 to 9;
R¹, R², R³, and R⁴ independently are C₁-C₆ alkyl, C₂-C₆ alkenyl, or C₂-C₆ alkynyl; or
R¹ and R²together with the carbon atom to which they are attached, or R³and R⁴ together with the carbon atom to which they are attached, or R¹ and R² together with the carbon atom to which they are attached and R³and R⁴together with the carbon atom to which they are attached, can complete a carbocyclic ring having from 3 to 6 carbons;
Y¹ and Y2 independently are COOH, CHO, tetrazole, or COOR⁵, wherein
R⁵ is C₁-C₆ alkyl, C₂-C₆ alkenyl, or C₂-C₆ alkynyl; and
wherein the alkyl, alkenyl, and alkynyl groups may be substituted with one or two groups selected from halo, hydroxy, C₁-C₆ alkoxy, and phenyl.

2. A method of inhibiting proinflammatory cytokine induced CRP production disorder in a subject, said method comprising co-administering to the subject having or susceptible to such proinflammatory cytokine induced CRP production, a therapeutically-effective amount of a COX-2 inhibitor or a pharmaceutically acceptable salt thereof and a therapeutically-effective amount of a substituted dialkyl ether compound of Formula I 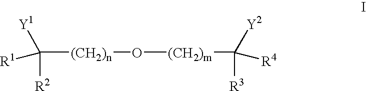
or a pharmaceutically acceptable salt thereof,
wherein:
n and m independently are integers of from 2 to 9;
R¹, R², R³, and R⁴ independently are C₁-C₆ alkyl, C₂-C₆ alkenyl, or C₂-C₆ alkynyl; or
R¹ and R² together with the carbon atom to which they are attached, or R³ and R⁴ together with the carbon atom to which they are attached, or R¹ and R² together with the carbon atom to which they are attached and R³ and R⁴together with the carbon atom to which they are attached, can complete a carbocyclic ring having from 3 to 6 carbons;
Y¹ and Y² independently are COOH, CHO, tetrazole, or COOR⁵, wherein
R⁵ is C₁-C₆ alkyl, C₂-C₆ alkenyl, or C₂-C₆ alkynyl; and
wherein the alkyl, alkenyl, and alkynyl groups may be substituted with one or two groups selected from halo, hydroxy, C₁-C₆ alkoxy, and phenyl.

3. The method of any one of claims 1-2, wherein said COX-2 inhibitor is
ABT-963;
valdecoxib;
BMS-347070;
celecoxib;
tilacoxib;
the compound of formula (B) 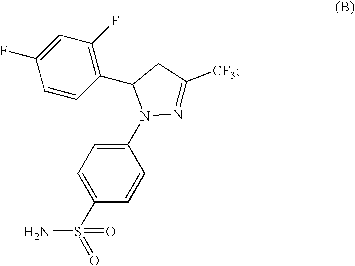
CS-502;
(6aR, 10aR)-3-(1,1-dimethylheptyl)-6a,7,10,10a-tetrahydro-1-hydroxy-6,6-dimethyl-6H-dibenzo[b,d]pyran-9-carboxylic acid;
CV-247;
2(5H)-Furanone, 5,5-dimethyl-3-(1-methylethoxy)-4-[4-(methylsulfonyl)phenyl]-(“DFP”);
carprofen;
deracoxib;
etoricoxib;
GW-406381;
tiracoxib;
meloxicam;
nimesulide;
2-(Acetyloxy)benzoic acid, 3-[(nitrooxy)methyl]phenyl ester;
lumiracoxib;
parecoxib;
P54;
rofecoxib;
revlMiD;
2,6-Bis(1,1-dimethylethyl)-4-[(E)-(2-ethyl-1,1-dioxo-5-isothiazolidinylidene)methyl]phenol;
5(R)-Thio-6-sulfonamide-3(2H)-benzofuranone;
N-[3-(Formylamino)-4-oxo-6-phenoxy-4H-1-benzopyran-7-yl]-methanesulfonamide; or
a pharmaceutically acceptable salt thereof.

4. The method of any one of claims 1-3, wherein said COX-2 inhibitor is carprofen, deracoxib, etoricoxib, lumiracoxib, parecoxib, rofecoxib, valdecoxib, celecoxib, or a pharmaceutically acceptable salt thereof.

5. The method of any one of claims 1-4, wherein said COX-2 inhibitor is carprofen, parecoxib, valdecoxib, celecoxib, or a pharmaceutically acceptable salt thereof.

6. The method of any one of claims 1-5, wherein said dialkyl ether is a compound of Formula I 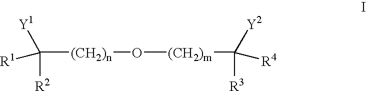
or a pharmaceutically acceptable salt thereof,
wherein:
n and m independently are integers of from 2 to 9;
R¹, R², R³, and R⁴ independently are C₁-C₆ alkyl; and
Y¹ and Y² independently are COOH or COOR⁵, wherein R⁵ is C₁-C₆ alkyl.

7. The method of any one of claims 1-6, wherein said dialkyl ether is 6-(5-carboxy-5-methyl-hexyloxy)-2,2-dimethyl-hexanoic acid, or a pharmaceutically acceptable salt thereof.

8. The method of any one of claims 1-7, wherein said dialkyl ether is 6-(5-carboxy-5-methyl-hexyloxy)-2,2-dimethyl-hexanoic acid, calcium salt.

9. The method of any one of claims 1-7, wherein said dialkyl ether is
6-(5-carboxy-5-methyl-hexyloxy)-2,2-dimethyl-hexanoic acid, calcium salt hydrate;
Crystal Form 1 of 6-(5-carboxy-5-methyl-hexyloxy)-2,2-dimethyl-hexanoic acid, calcium salt; or
Crystal Form 2 of 6-(5-carboxy-5-methyl-hexyloxy)-2,2-dimethyl-hexanoic acid, calcium salt.
